(19) United States
(12) Patent Application Publication
(10) Pub. No.: US 2022/0125989 A1
(43) Pub. Date: Apr. 28, 2022

(54) BACTERICIDAL COATING COMPOSITIONS AND METHODS USING SAME

(71) Applicant: Rowan University, Glassboro, NJ (US)

(72) Inventors: Jeffrey Hettinger, Cherry Hill, NJ (US); Gregory A. Caputo, Glassboro, NJ (US); Lei Yu, Cherry Hill, NJ (US)

(21) Appl. No.: 17/507,951

(22) Filed: Oct. 22, 2021

Related U.S. Application Data
(60) Provisional application No. 63/104,241, filed on Oct. 22, 2020.

(51) Int. Cl. A61L 27/06; A61L 27/04; A61L 27/08; A61N 1/05

(52) U.S. Cl. CPC A61L 27/06; A61N 1/05; A61L 27/08; A61L 27/047

(57) ABSTRACT

The present disclosure relates in part to coating compositions comprising a bactericidal layer further comprising a bactericidal element and a columnar microstructure, which exerts bactericidal activity toward proximal and distal bacteria within an electrolyte solution (i.e. blood or other bodily fluid). The present disclosure further relates to coating compositions stably adhered to an electrode, which exerts bactericidal activity toward proximal and distal bacteria within an electrolyte solution upon application of an electric potential to the underlying electrode without a loss in efficiency of charge transfer.

### CROSS-REFERENCE TO RELATED APPLICATIONS

The present application claims priority under 35 U.S.C. § 119(e) to U.S. Provisional Patent Application No. 63/104,241, filed Oct. 22, 2020, which is incorporated herein by reference in its entirety.

### BACKGROUND OF THE INVENTION

Millions of people worldwide have had cardiac devices implanted, including pacemakers and defibrillators, with approximately one million pacemakers and 300,000 cardiac defibrillators implanted each year. The number of individuals with implanted cardiac devices is expected to increase as the average age of the population of many industrialized nations continues to increase. While the majority of individuals with an implanted cardiac device do not experience difficulties associated with the device, in approximately 1.5% of cases the subject develops an infection associated with the implanted device. Under circumstances in which the infected subject requires hospitalization, the mortality rate has a range of 10-15%. Furthermore, infection and mortality rates can be substantially higher for subjects with pre-existing conditions and/or for subjects requiring revisional surgery.

Thus, there is a need in the art for a bactericidal coating that can be used on implanted devices, including but not limited to implanted electrodes. The present disclosure addresses this need.

### SUMMARY

In some embodiments, the instant specification is directed to the non-limiting exemplary embodiments below. The numbering of the exemplary embodiments is not to be construed as designating levels of importance:

Embodiment 1 provides a composition comprising a substrate at least partially coated with a bactericidal coating, the composition comprising; a substrate having a surface; and a bactericidal layer comprising a bactericidal metal element and a columnar microstructure, wherein the bactericidal layer comprises at least one of the following:
(a) a multilayer having a first layer comprising the bactericidal metal element, wherein the first layer is stably adhered to at least one portion of the substrate surface; and a second layer comprising the columnar microstructure, wherein the second layer is stably adhered to at least one portion of the first layer; or
(b) a composite comprising the bactericidal metal element and the columnar microstructure, wherein the composite is stably adhered to at least one portion of the substrate surface.
For (a): Optionally, the first layer or the bactericidal metal element comprises a second columnar microstructure.
For (a): Optionally, the substrate comprises a third columnar structure, such as a third columnar structure formed from a metal nitride, a carbon nanotube (the metal nitride and/or the carbon nanotube are the same as or different from those as described elsewhere in Embodiments 5 or 6), or a second bactericidal metal element (which is the same as or different from the bactericidal metal element in the first layer). For (a) and (b): Optionally, (a) and (b) are combined such that the bactericidal layer comprises the multilayer of (a) in which the first layer comprises a composite of (b).

Embodiment 2 provides the composition of Embodiment 1, wherein the bactericidal metal element is at least one selected from the group consisting of Ag, Cu, Zn, Ni, Sn, Au, and Co.

Embodiment 3 provides the composition of any one of Embodiments 1-2, wherein the bactericidal metal element comprises Ag.

Embodiment 4 provides the composition of any one of Embodiments 1-2, wherein the bactericidal metal element comprises Cu. Embodiment 4a provides the composition of any one of Embodiments 1-3, wherein the bactericidal metal element comprises Cu and Ag.

Embodiment 5 provides the composition of any one of Embodiments 1-4, wherein the columnar microstructure comprises a metal nitride or carbon nanotube.

Embodiment 6 provides the composition of Embodiment 5, wherein the metal nitride is at least one selected from the group consisting of titanium nitride (TiN), zirconium nitride (ZrN), tantalum nitride (TaN), and vanadium nitride (VN).

Embodiment 7 provides the composition of Embodiment 6, wherein the metal nitride is TiN.

Embodiment 8 provides the composition of Embodiment 6, wherein the metal nitride is ZrN.

Embodiment 9 provides the composition of any one of Embodiments 1-8, wherein, if the bactericidal metal element is Ag, the columnar microstructure is not titanium nitride (TiN).

Embodiment 10 provides the composition of any one of Embodiments 1-9, wherein the substrate is selected from the group consisting of intramedullary nails, pins, rods, plates, screws, artificial joints, artificial heart components, prosthetic blood vessels, catheters, stents, wound dressings, surgical stitching fibers, and pharmaceutical depots.

Embodiment 11 provides the composition of any one of Embodiments 1-10, wherein the substrate surface comprises titanium or stainless steel. Embodiment 11a provides the composition of any one of Embodiments 1-10, wherein the substrate surface comprises platinum, gold, a platinum-containing alloy, a gold containing alloy, or combinations thereof.

Embodiment 12 provides the composition of any one of Embodiments 1-11, wherein the first layer comprising the bactericidal metal element has a thickness of 0.75 μm.

Embodiment 13 provides the composition of any one of Embodiments 1-12, wherein the second layer comprising the columnar microstructure has a thickness of about 0.50 μm to about 2.00 μm.

Embodiment 14 provides the composition of any one of Embodiments 1-13, wherein the second layer comprising the columnar microstructure has a thickness selected from the group consisting of about 0.50 μm, about 0.75 μm, about 0.800 μm, about 1.00 μm, about 1.20 μm, about 1.60 μm, and about 2.00 μm.

Embodiment 15 provides the composition of any one of Embodiments 1-14, wherein the multilayer is synthesized by a method comprising DC magnetron sputtering the bactericidal metal element onto at least a portion of the substrate's surface and DC reactive magnetron sputtering the columnar microstructure layer onto a portion of the substrate's surface that is coated by the bactericidal metal element.

Embodiment 16 provides the composition of any one of Embodiments 1-15, wherein the bactericidal metal element comprises a percentage of the composite bactericidal layer which is selected from the group consisting of about 1% to about 5%, about 5% to about 10%, about 10% to about 15%, about 15% to about 20%, about 20% to about 25%.

Embodiment 17 provides the composition of any one of Embodiments 1-16, wherein the bactericidal metal element comprises a percentage of the composite bactericidal layer which is selected from the group consisting of 9%, 13%, 20%, 22%, and 24%.

Embodiment 18 provides the composition of any one of Embodiments 1-17, wherein the bactericidal metal element comprises 13% of the composite bactericidal layer and the bactericidal metal element is Ag.

Embodiment 19 provides the composition of any one of Embodiments 1-18, wherein the bactericidal metal element comprises a percentage of the composite bactericidal layer which is selected from the group consisting of about 5%, about 6%, about 16%, about 17%, and about 18%.

Embodiment 20 provides the composition of any one of Embodiments 1-19, wherein the bactericidal metal element comprises about 5% of the composite bactericidal layer and the bactericidal metal element is Cu.

Embodiment 21 provides the composition of any one of Embodiments 1-20, wherein the composite coating has a thickness in the range of about 0.5 μm to about 30 μm, such as but not limited to about 0.6 μm, about 0.7 μm, about 0.8 μm, about 0.9 μm, about 1.0 μm, about 1.2 μm, about 1.4 μm, about 1.6 μm, about 1.8 μm, about 2.0 μm, about 2.5 μm, about 3.0 μm, about 4 μm, about 5 μm, about 6 μm, about 8 μm, about 10 μm, about 12 μm, about 14 μm, about 16 μm, about 18 μm, about 20 μm, about 22 μm, about 25 μm, or about 30 μm.

Embodiment 22 provides the composition of any one of Embodiments 1-21, wherein the composite bactericidal layer is synthesized by co-deposition of the bactericidal metal element and Ti or Zr onto at least a portion of the substrate's surface with a partial pressure of Na. Embodiment 22a provides the composition of any one of Embodiments 1-21, wherein the composite is synthesized by co-deposition of one or more bactericidal metal elements and one or more transitions metals (such as Ti and Zr) onto at least a portion of the substrate's surface with a partial pressure of N₂

Embodiment 23 provides the composition of any one of Embodiments 1-22, wherein the composition is in contact with an electrolyte solution.

Embodiment 24 provides the composition of any one of Embodiments 1-23, wherein the electrolyte solution contains at least one bacterial species.

Embodiment 25 provides the composition of Embodiment 24, wherein the bacterial species is at least one of S. aureus and P. aeruginosa.

Embodiment 26 provides the composition of any one of Embodiments 24-25, wherein proximal and distal bacterial growth within the electrolyte solution is reduced or eliminated.

Embodiment 27 provides an electrode article that is implantable in a subject, the article comprising: an electrode substrate having a surface; and a bactericidal layer comprising a bactericidal metal element and a columnar microstructure, wherein the bactericidal layer comprises at least one of the following:
(a) a multilayer having a first layer comprising the bactericidal metal element, wherein the first layer is stably adhered to at least one portion of the substrate surface; and a second layer comprising the columnar microstructure, wherein the second layer is stably adhered to at least one portion of the first layer; or
(b) a composite comprising the bactericidal metal element and the columnar microstructure, wherein the composite is stably adhered to at least one portion of the substrate surface.
   wherein ions of the bactericidal metal element are released by application of an electrical potential to the electrode.
For (a): Optionally, the first layer or the bactericidal metal element comprises a second columnar microstructure.
For (a): Optionally, the substrate comprises a third columnar structure, such as a third columnar structure formed from a metal nitride, a carbon nanotube (the metal nitride and/or the carbon nanotube are the same as or different from those as described below in Embodiments 31 or 32), or a second bactericidal metal element (which is the same as or different from the bactericidal metal element in the first layer). For (a) and (b): Optionally, (a) and (b) are combined such that bactericidal layer has the multilayer of (a) in which the first layer comprises a composite of (b).

Embodiment 28 provides the article of Embodiment 27, wherein the bactericidal metal element is at least one selected from the group consisting of Ag, Cu, Zn, Ni, Sn, Au, and Co.

Embodiment 29 provides the article of any one of Embodiments 27-28, wherein the bactericidal metal element comprises Ag.

Embodiment 30 provides the article of any one of Embodiments 27-28, wherein the bactericidal metal element comprises Cu. Embodiment 30a provides the article of any one of Embodiments 27-29, wherein the bactericidal metal element comprises Cu and Ag.

Embodiment 31 provides the article of any one of Embodiments 27-30, wherein the columnar microstructure layer comprises a metal nitride or carbon nanotube.

Embodiment 32 provides the article of any one of Embodiments 27-31, wherein the metal nitride is at least one selected from the group consisting of titanium nitride (TiN), zirconium nitride (ZrN), tantalum nitride (TaN), and vanadium nitride (VN).

Embodiment 33 provides the article of Embodiment 32, wherein the metal nitride is TiN.

Embodiment 34 provides the article of Embodiment 32, wherein the metal nitride is ZrN.

Embodiment 35 provides the article of any one of Embodiments 27-34, wherein the substrate surface comprises titanium or stainless steel. Embodiment 35a provides the article of any one of Embodiments 27-34, wherein the substrate surface comprises platinum, gold, a platinum-containing alloy, a gold containing alloy, or combinations thereof.

Embodiment 36 provides the article of any one of Embodiments 27-35, wherein the electrode comprises an implantable medical device selected from the group consisting of a pacemaker, cardioverter defibrillator, retinal implant, phrenic nerve stimulator, glucose biosensor, cochlear implant, and an electrical stimulator for pain relief management, Parkinson's disease, and/or epilepsy.

Embodiment 37 provides the article of any one of Embodiments 27-36, wherein the first layer comprising the bactericidal metal element has a thickness of 0.75 μm.

Embodiment 38 provides the article of any one of Embodiments 27-37, wherein the second layer comprising the columnar microstructure has a thickness of about 0.50 μm to about 2.00 μm.

Embodiment 39 provides the article of any one of Embodiments 27-38, wherein the second layer comprising the columnar microstructure has a thickness selected from the group consisting of 0.50 μm, 0.75 μm, 0.800 μm, 1.00 μm, 1.20 μm, 1.60 μm, and 2.00 μm.

Embodiment 40 provides the article of any one of Embodiments 27-39, wherein the mutilayer is synthesized by DC magnetron sputtering the bactericidal metal element onto at least a portion of the substrate's surface and DC reactive magnetron sputtering the columnar microstructure layer onto a portion of the substrate's surface that is coated by the bactericidal metal element.

Embodiment 41 provides the article of any one of Embodiments 27-40, wherein the bactericidal metal element comprises a percentage of the composite bactericidal layer which is selected from the group consisting of about 1% to about 5%, about 5% to about 10%, about 10% to about 15%, about 15% to about 20%, about 20% to about 25%.

Embodiment 42 provides the article of any one of Embodiments 27-41, wherein the bactericidal metal element comprises a percentage of the composite bactericidal layer which is selected from the group consisting of 9%, 13%, 20%, 22%, and 24%.

Embodiment 43 provides the article of any one of Embodiments 27-42, wherein the bactericidal metal element comprises 13% of the composite bactericidal layer and the bactericidal metal element is Ag.

Embodiment 44 provides the article of any one of Embodiments 27-43, wherein the bactericidal metal element comprises a percentage of the composite bactericidal layer which is selected from the group consisting of about 5%, about 6%, about 16%, about 17%, and about 18%.

Embodiment 45 provides the article of any one of Embodiments 27-44, wherein the bactericidal metal element comprises about 5% of the composite bactericidal layer and the bactericidal metal element is Cu.

Embodiment 46 provides the article of any one of Embodiments 27-45, wherein the composite coating has a thickness in the range of about 0.5 μm to about 30 μm, such as but not limited to about 0.6 μm, about 0.7 μm, about 0.8 μm, about 0.9 μm, about 1.0 μm, about 1.2 μm, about 1.4 μm, about 1.6 μm, about 1.8 μm, about 2.0 μm, about 2.5 μm, about 3.0 μm, about 4 μm, about 5 μm, about 6 μm, about 8 μm, about 10 μm, about 12 μm, about 14 μm, about 16 μm, about 18 μm, about 20 μm, about 22 μm, about 25 μm, or about 30 μm.

Embodiment 47 provides the article of any one of Embodiments 27-46, wherein the composite is synthesized by co-deposition of the bactericidal metal element and Ti or Zr onto at least a portion of the substrate's surface with a partial pressure of N₂. Embodiment 47a provides the article of any one of Embodiments 27-46, wherein the composite is synthesized by co-deposition of one or more bactericidal metal elements and one or more transitions metals (such as Ti and Zr) onto at least a portion of the substrate's surface with a partial pressure of N₂.

Embodiment 48 provides the article of any one of Embodiments 27-47, wherein the composition is in contact with an electrolyte solution.

Embodiment 49 provides the article of any one of Embodiments 27-48, wherein the electrolyte solution contains at least one bacterial species.

Embodiment 50 provides the article of Embodiment 49, wherein the bacterial species is at least one of S. aureus and P. aeruginosa.

Embodiment 51 provides the article of any one of Embodiments 49-50, wherein proximal and distal bacterial growth within the electrolyte solution is reduced or eliminated.

Embodiment 52 provides the article of any one of Embodiments 27-51, wherein an electrical potential is applied to the electrode.

Embodiment 53 provides the article of Embodiment 52, wherein the electrical potential applied to the electrode ranges from about +0.1 V to about +0.8 V.

Embodiment 54 provides the article of any one of Embodiments 52-53, wherein ions of the bactericidal metal element are released in the electrolyte solution.

Embodiment 55 provides the article of any one of Embodiments 52-54, wherein proximal and distal bacterial growth within the electrolyte solution is reduced or eliminated.

Embodiment 56 provides the article of any one of Embodiments 27-55, wherein the bactericidal layer does not decrease the efficiency of underlying electrode.

Embodiment 57 provides the article of any one of Embodiments 27-56, wherein the subject is a mammal.

Embodiment 58 provides the article of Embodiment 57, wherein the mammal is a human.

Embodiment 59 provides the composition of any one of Embodiments 1-26, wherein the bactericidal layer comprises the composite, and wherein an amount of the bactericidal metal element is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% (mol/mol) based on a total moles of metal elements for forming the bactericidal layer.

Embodiment 60 provides the composition of any one of Embodiments 1-26 and 59, wherein the bactericidal layer comprises the composite, and a deposition pressure of the bactericidal metal element and a deposition pressure of a metal element for forming the columnar microstructure are each independently about 1 mTorr, about 2 mTorr, about 3 mTorr, about 4 mTorr, about 5 mTorr, about 10 mTorr, about 15 mTorr, about 20 mTorr, about 25 mTorr, about 30 mTorr, about 40 mTorr, about 50 mTorr, about 60 mTorr, about 70 mTorr, about 80 mTorr, about 90 mTorr, or about 100 mTorr.

Embodiment 61 provides the article of any one of Embodiments 27-58, wherein the bactericidal layer comprises the composite, and wherein an amount of the bactericidal metal element is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% (mol/mol) based on a total moles of metal elements for forming the bactericidal layer.

Embodiment 62 provides the article of any one of Embodiments 27-58 and 61, wherein the bactericidal layer comprises the composite, and a deposition pressure of the bactericidal metal element and a deposition pressure of a metal element for forming the columnar microstructure are each independently about 1 mTorr, about 2 mTorr, about 3 mTorr, about 4 mTorr, about 5 mTorr, about 10 mTorr, about 15 mTorr, about 20 mTorr, about 25 mTorr, about 30 mTorr, about 40 mTorr, about 50 mTorr, about 60 mTorr, about 70 mTorr, about 80 mTorr, about 90 mTorr, or about 100 mTorr.

Embodiment 63 provides the article of any one of Embodiments 27-58, 61 and 62, wherein the article further comprises a power source, such as a battery or a power supply.

### BRIEF DESCRIPTION OF THE FIGURES

The drawings illustrate generally, by way of example, but not by way of limitation, various embodiments of the present application.

FIG. 1 shows a schematic of a columnar microstructure with released Ag ions from the correlated pores between columns killing bacteria in solution.

FIG. 2 shows an image of an implanted cardiac device (pacemaker), wherein an electrode is positioned within the heart at the site of applied potential.

FIG. 3 shows a diagnostic tool possessing flexible wings with electrodes thereupon which can serve as a position for bactericidal coating.

FIG. 4 shows a schematic of a Ag/TiN bilayer coating adhered to a substrate and the relative thicknesses of each of the TiN and Ag constituent layers.

FIGS. 5A-5D show SEM images of TiN coatings deposited on the surface of a 0.75 μm Ag layer, wherein the TiN coatings have a thickness of: 0.50 μm (FIG. 5A); 0.75 μm (FIG. 5B); 1.00 μm (FIG. 5C); and 2.00 μm (FIG. 5D).

FIG. 6 shows a TiN coating cross-section milled with a gallium ion beam.

FIG. 7 shows overlaid X-ray diffraction (XRD) spectra of Ag/TiN bilayer coatings: (A) 0.75 μm Ag and 0.50 μm TiN; (B) 0.75 μm Ag and 0.75 μm TiN; (C) 0.75 μm Ag and 1.00 μm TiN; and (D) 0.75 μm Ag and 2.00 μm TiN.

FIG. 8 shows an SEM image of a fractured cross-section of a Ag/TiN bilayer coating. The red arrows indicate the interface between the Ag and TiN layers.

FIGS. 9A-9B show SEM images of the microstructure of cross-sections of a TiN coating at low-magnification (FIG. 9A); and high-magnification (FIG. 9B).

FIGS. 10A-10B show cyclic voltammograms of utilizing Ag/TiN bilayer coatings wherein the thickness of the TiN layer is varied. FIG. 10A shows the cyclic voltammogram with applied potential between −0.6 V and +0.8 V. FIG. 10B shows the cyclic voltammogram with applied potential between 0.0 V an +0.8 V.

FIG. 11 provides the results of inductively coupled plasma mass-spectrometry (ICP-MS) analysis of electrolyte solutions following electrochemical cycling of several Ag/TiN bilayer coatings. For each Ag/TiN bilayer coating the thickness of the TiN layer was varied, and the Ag ion concentration is provided for each Ag/TiN bilayer sample after applied potential between −0.6 V and +0.8 V (left bar), and 0.0 and +0.8 V (right bar).

FIG. 12 shows an SEM image of a fractured Ag/TiN coating after cyclic voltammetry was performed.

FIGS. 13A-13D show SEM images of TiN coatings deposited on the surface of a 0.75 μm Cu layer, wherein the TiN coatings have a thickness of: 0.50 μm (FIG. 13A); 0.80 μm (FIG. 13B); 1.20 μm (FIG. 13C); and 1.60 μm (FIG. 13D).

FIG. 14 shows overlaid X-ray diffraction (XRD) spectra of Cu/TiN bilayer coatings: (A) 0.75 μm Cu and 0.50 μm TiN; (B) 0.75 μm Cu and 0.80 μm TiN; (C) 0.75 μm Cu and 1.20 μm TiN; and (D) 0.75 μm Cu and 1.60 μm TiN.

FIG. 15 shows a cyclic voltammogram of utilizing Cu/TiN bilayer coatings wherein the thickness of the TiN layer is varied with applied potential between −0.6 V and +0.8 V.

FIG. 16 provides the results of inductively coupled plasma mass-spectrometry (ICP-MS) analysis of electrolyte solutions following electrochemical cycling of several Cu/TiN bilayer coatings. For each Cu/TiN bilayer coating the thickness of the TiN layer was varied, and the Cu ion concentration is provided for each Cu/TiN bilayer sample after applied potential between −0.6 V and +0.8 V (triplicate experiments).

FIGS. 17A-17B provide an SEM images demonstrating the role of high concentrations of Ag in the TiN matrix. FIG. 17A: 10% Ag/TiN composite (scale bar: 2 μm). FIG. 17B: 26% Ag/TiN composite (scale bar: 5 μm).

FIGS. 18A-18C are SEM images showing Ag particulates forming on the surface of the Ag/TiN coatings a few days after deposition. FIG. 18A shows micron scale Ag clusters on the surface of a Ag/TiN composite coating. FIG. 18B shows Ag particulates forming on the surface of a Ag/TiN composite coating comprising 9% Ag (scale bar: 2 μm). FIG. 18C shows Ag particulates forming on the surface of a Ag/TiN composite coating comprising 20% Ag (scale bar: 2 μm).

FIG. 19 provides an SEM image of a fractured cross-section of a Ag/TiN composite coating illustrating the decoration of the columnar interfaces with Ag nanoparticles. Each column is approximately 200 nm and the Ag nanoparticles are typically less than 50 nm in size (Ag particulates indicated with arrows).

FIG. 20 shows a schematic of the Ag nanoparticles that are expelled from the TiN matrix.

FIGS. 21A-21D provide SEM images of Ag/ZrN composite coatings prepared with varying deposition powers (scale bar: 500 nm). FIG. 21A shows a Ag/ZrN composite coating.

FIG. 21B shows a Ag/ZrN composite coating deposited at high power. FIG. 21C shows a Ag/ZrN composite coating after a few days. FIG. 21D shows a Ag/ZrN composite coating with high Ag content.

FIGS. 22A-22B provide bar graphs showing inhibition of S. aureus biofilm formation in the presence of Ag/TiN coated metal discs. Stainless steel (SS) or titanium (Ti) discs were coated with Ag/TiN compositions as indicated by sample number. Strong biofilm inhibition (OD₅₇₀<0.2); weak biofilm inhibition (0.2<OD₅₇₀<1); non-biofilm inhibition (OD₅₇₀>1). FIG. 22A provides the results using samples 6-7 and 10-15. FIG. 22B provides the results using samples 16-21.

FIGS. 23A-23B provide bar graphs showing inhibition of P. aeruginosa biofilm formation in the presence of Ag/TiN coated metal discs. Stainless steel (SS) or titanium (Ti) discs were coated with Ag/TiN compositions as indicated by sample number. Non-adherent (OD₅₇₀≤OD₅₇₀ control); weakly adherent (OD₅₇₀ control<OD₅₇₀≤2×OD₅₇₀ control); strongly adherent (4×OD₅₇₀ control<OD₅₇₀). FIG. 23A provides the results using samples 6-7 and 10-15. FIG. 23B provides the results using samples 16-21.

FIG. 24 is a bar graph showing inhibition of biofilm growth of S. aureus on the surface of samples coated with Cu/TiN compositions. The two horizontal lines indicate the point of differentiation between degrees of biofilm inhibition exhibited by different coatings. Strong biofilm inhibition (OD₅₇₀<0.2); weak biofilm inhibition (0.2<OD₅₇₀<1); non-biofilm inhibition (OD₅₇₀>1).

FIGS. 25A-25E provide the results of growth kinetics studies of S. aureus in the presence of Ag/TiN coating metal discs. Stainless steel (SS) or titanium (Ti) discs were coated with Ag/TiN compositions as indicated by sample number. FIG. 25A provides the results using samples 6-7 and 10. FIG. 25B provides the results using samples 11-13. FIG. 25C provides the results using samples 14-17. FIG. 25D provides the results using samples 18-20. FIG. 25E provides the results using samples 22-24.

FIGS. 26A-26E provide the results of growth kinetics studies of P. aeuroginosa in the presence of Ag/TiN coated metal discs. Stainless steel (SS) or titanium (Ti) discs were coated with Ag/TiN compositions as indicated by sample number. FIG. 26A provides the results using samples 6-7 and 10. FIG. 26B provides the results using samples 11-13. FIG. 26C provides the results using samples 14-17. FIG. 26D provides the results using samples 18-20. FIG. 26E provides the results using samples 22-24.

FIGS. 27A-27B provide the results of growth kinetics studies of S. aureus (FIG. 27A) and P. aeruginosa (FIG. 27B) in the presence of Cu/TiN coated metal discs. Stainless steel (SS) discs were coated with Cu/TiN compositions as indicated by sample number. FIG. 27A provides the results of S. aureus growth in the presence of samples 1-4. FIG. 27B provides the results of P. aeruginosa growth in the presence of samples 1-4.

FIGS. 28A-28B provide images of bacterial growth of P. aeruginosa grown on a LB agar plate, incubated for 12-17 hours at 37° C. after plating. Bacterial samples plated on the LB agar plate were first subjected to cyclic voltammetry (CV) experiments with Ag/TiN coated electrodes. FIG. 28A provides the image wherein the plated sample was taken from a CV experiment with a total volume of 20 mL immediately after the CV experiment. FIG. 28B provides the image wherein the plated sample was taken from a CV experiment with a total volume of 1 mL immediately after the CV experiment.

FIG. 29 shows an image showing pillars of TiN having pores therebetween, in accordance with some embodiments. The pores allow ions, such as ions in the body fluids (such as blood plasma), as well as ions generated by bactericidal metal element to pass.

FIG. 30 shows scanning electron microscopy images of TiN with various amount of zinc (upper left: 0%, upper right: 5.5%, lower left: 28% and lower right: 38%), in accordance with some embodiments. All panels in FIG. 30 except for the lower right panel are at the same magnification. The features shown in the lower right panel are larger and therefore the magnification was reduced by about a factor of 10. The cube-corners decorating the surface are lost between images of the lower left and lower right panels.

FIG. 31 shows XRD spectra for TiN samples made with varying amounts of Zn, in accordance to some embodiments. As the figure shows, there is a sharp reduction in the size of the (111) and (222) peaks in the 38% Zn material as well as an emergence of the TiN (002) peak. These changes suggest a loss of texture in the sample which implies that the materials are becoming more disorganized and crystallites are becoming randomly oriented.

FIG. 32 shows the XRD spectra for TiN coatings including different amounts of Cu, in accordance with some embodiments.

FIG. 33 shows the XRD spectra for TiN coatings including different amounts of Au, in accordance with some embodiments.

FIG. 34 shows the XRD spectra for TiN coatings deposited with the addition of various amount of Ag at about 0.67 Pa (5 mTorr, left panel) and about 4.00 Pa (30 mTorr, right panel), in accordance with some embodiments.

FIG. 35 depicts the plot of the estimated concentrations of metal ions at various potentials, in accordance with some embodiments. As shown in the figure, at approximately 0.65V, the concentration of Ag-ions released increases and continue to increase until the potential reaches potentials greater than 0.90V. The first set of data (with lower metal ion concentration between 40 to 50 minutes) is from a sample that contained approximately 2% Ag while the second set of data (with higher metal ion concentration between 40 to 50 minutes) is from a sample with approximately 30% Ag.

FIG. 36 shows an SEM cross-sectional image showing TiN+TiNAg+TiN tri-layer coating, in accordance with some embodiments. The SEM image demonstrates that a multilayer coating can be synthesized with varying bactericidal element in different layers.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise.

Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

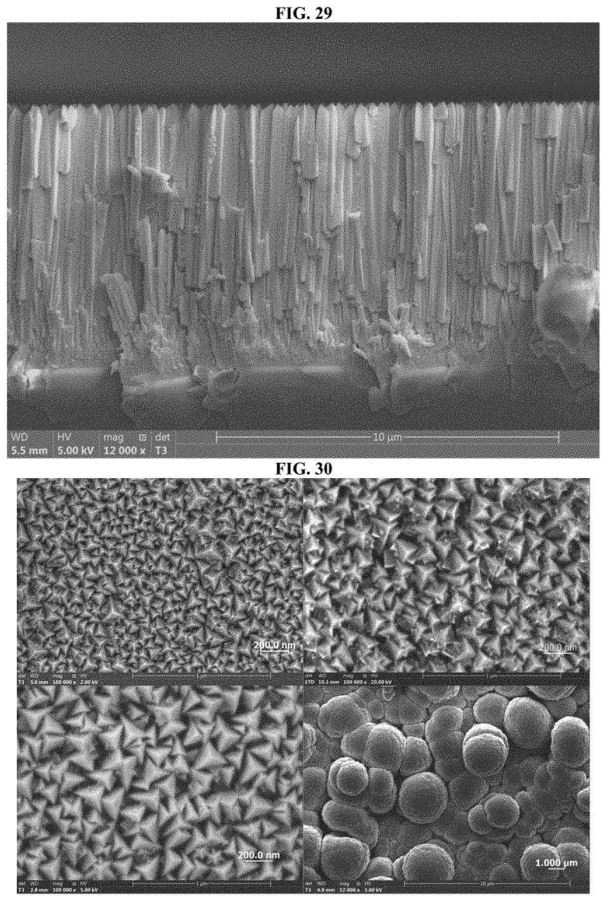

Pacemaker electrodes are often coated with titanium nitride (TiN) to enhance the performance of the implanted electrode for pacing and recording applications. The TiN coatings have columnar microstructure formed by TiN (FIG. 29). Other materials that form similar columnar microstructures include, but are not limited to, carbon nanotubes and other metal nitrides, non-limiting examples including zirconium nitride (ZrN), tantalum nitride (TaN), and vanadium nitride (VN).

One feature of such coatings is the pore formed by the voids between the columnar microstructure (FIG. 29). Such pores allow ion-containing solutions (i.e., blood plasma/phosphate buffered saline) to penetrate and exchange charge by induction, thereby exchanging electric field.

Many implantable devices have bactericidal coatings to decrease the risk of infection, with such coatings often comprising small organic molecules, such as antibiotics, which are embedded in the surface of the implant and are gradually released. However, bactericidal coatings including small organic compounds can be problematic as the use of such compounds tends to lead to resistances, such as antibiotic resistance. Additionally, antibiotic coatings often reduce the overall surface area of the electrode by blocking pores, thereby decreasing the charge transfer efficiency of the electrode.

It was conceived that the pores between the columnar microstructures would also allow for bactericidal elements (such as bactericidal metal elements) beneath, around, or within the columnar microstructures to be accessible such that the bactericidal elements are able to kill bacteria adhered or in close proximity to the coatings and therefore suppresses biofilm formation. The pores add to the effective surface area of the electrode, allowing the overall geometric electrode size to be reduced while retaining desirable charge exchange performance. The pores would also allow for the diffusion of ions of bactericidal elements to the bacteria in the biological solutions not immediately adjacent to the columnar microstructures.

Accordingly, in some aspects, the present invention is directed to a bactericidal coating (also referred to as “bactericidal layer” or “coating”) having resistance to bacteria, such as resistance to the formation of microfilm by bacteria thereon. In some embodiments, the bactericidal layer includes a columnar microstructure, as well as a bactericidal element beneath, around, and/or within the columnar microstructure. In some embodiments, when the bactericidal coating is exposed to a solution, such as blood plasma or other body fluids, ions of the bactericidal element are released and enriched in proximity to the bactericidal coating, such as in the voids between multiple columnar microstructures. In some embodiments, ions of the bactericidal element are released in response to an application of electrical potential to the bactericidal layer.

In some aspects, the present invention is directed to a composition having resistance to bacteria, such as resistance to the formation of microfilm by bacteria thereon. In some embodiments, the composition includes a substrate having a surface, and a bactericidal layer. In some embodiments, the bactericidal layer includes a columnar microstructure, as well as a bactericidal element beneath, around, and/or within the columnar microstructure. In some embodiments, when the composition is exposed to a solution, such as blood plasma or other body fluids, ions of the bactericidal element are released and enriched in proximity to the composition, such as in the voids between multiple columnar microstructures. In some embodiments, ions of the bactericidal element are released in response to an application of electrical potential to the bactericidal layer.

In some aspects, the present invention is directed to an electrode article having resistance to bacteria, such as resistance to the formation of microfilm by bacteria thereon. In some embodiments, the electrode article includes an electrode substrate having a surface, and a bactericidal layer. In some embodiments, the bactericidal layer includes a columnar microstructure, as well as a bactericidal element beneath, around, and/or within the columnar microstructure. In some embodiments, when the electrode article is exposed to a solution, such as blood plasma or other body fluids, ions of the bactericidal element are released and enriched in proximity to the electrode article, such as in the voids between multiple columnar microstructures. In some embodiments, ions of the bactericidal element are released in response to an application of electrical potential to the bactericidal layer.

It is worth noting that, although the instant specification sometimes uses artificial pacemaker as a non-limiting illustrative example, the composition and the electrode article as described herein are not limited thereto. One of ordinary skill in the art would understand that the desirable features of the instant composition and electrode article make the composition and electrode article suitable for all implantable devices, such as pacemakers, cardioverter defibrillators, retinal implants, phrenic nerve stimulators, glucose biosensors, cochlear implants, or electrical stimulators for pain relief management/Parkinson's disease/epilepsy, as well as other non-implantable articles where bactericidal effects and/or biofilm resistance are desired.

### Definitions

Certain abbreviations used herein follow: CFU, colony forming units; CV, cyclic voltammetry; DC, direct current; EDS, energy dispersive spectroscopy; ICP-MS, inductively coupled plasma mass spectrometry; OD, optical density; PVD, physical vapor deposition; SEM, scanning electron microscopy; SS, stainless steel; TaN, tantalum nitride; TiN, titanium nitride; VN, vanadium nitride; XRD, x-ray diffraction; ZrN, zirconium nitride.

The term “about” as used herein can allow for a degree of variability in a value or range, for example, within 10%, within 5%, or within 1% of a stated value or of a stated limit of a range, and includes the exact stated value or range.

The term “bactericidal” as used herein refers to a substance or structure which kills bacteria including, but not limited to metals, disinfectants, antiseptics, or antibiotics.

The term “multilayer coating” as used herein refers to a substance which is stably adhered to at least a portion of the surface of an object, which is referred to as the substrate, wherein the substance comprises two or more discrete layers of differing composition which are stably adhered to each other. The term “bilayer coating” as used herein refers to a substance which is stably adhered to the surface of an object, which is referred to as the substrate, wherein the substance comprises two discrete layers of differing composition which are stably adhered to one another.

The term “columnar microstructure” as used herein refers to the morphological features of a material observable at the micrometer (μm)) scale, wherein the structure comprises a low density or porous network and an array of parallel uniform-sized rods of higher density which are perpendicularly arranged with respect to the basal plane of the substrate upon which the material is deposited. The pillars normally have an average width ranging from about 50 μm to about 750 μm, such as about 50 μm, about 60 μm, about 70 μm, about 80 μm, about 90 μm, about 100 μm, about 120 μm, about 140 μm, about 160 μm, about 180 μm, about 200 μm, about 250 μm, about 300 μm, about 400 μm, about 500 μm, or about 750 μm. The pores formed by the voids between the pillars normally have an average diameter ranging from 20 nm to 300 nm, such as about 50 nm, about 60 nm, about 70 nm, about 80 nm, about 90 nm, about 100 nm, about 120 nm, about 140 nm, about 160 nm, about 180 nm, about 200 nm, about 250 nm, about 300 nm.

The term “composite coating” as used herein refers to a substance which is stably adhered to the surface of an object, referred to as the substrate, wherein the substance comprises at least two constituent materials that are not discretely divided into two layers.

The term “distal growth” as used herein refers to the growth of bacteria in the electrolyte solution which are located at a distance sufficiently far from the coating-electrolyte solution interface as to avoid direct contact with the coating composition.

The term “electrode” as used herein refers to any electrically conductive element used to contact a non-metallic component of a circuit and may refer to an anode and/or cathode.

The term “electrolyte solution” as used herein refers to an electrically conductive liquid comprising a solvent, non-limiting examples including water, and an ionizable substance, referred to as the electrolyte. In certain embodiments, the term may refer to blood, growth media, and/or other liquid biological samples.

The term “magnetron sputtering” as used herein refers to a method of physical vapor deposition (PVD), wherein a thin film is deposited onto the surface of a substrate. The process involves ion bombardment of a source material, the target, resulting in sputtering of the target material which is deposited on the surface of the substrate. Magnetron sputtering employs a magnetron source in which positive ions present in the plasma of a magnetically enhanced glow discharge bombard the target, which can be powered by direct current (DC), but the present invention is not limited thereto. One of ordinary skill in the art would understand that the PVD and sputtering as described herein can also be carried out with radio frequency (RF), pulsed DC, evaporation, and the like, and these PVD/sputtering methods are within the scope of the present invention. In certain embodiments the present invention relates to compositions prepared by reactive sputtering, such as a reactive sputtering process in which the magnetron sputtering process is coupled with a chemical reaction, thereby depositing material other than the target source upon the substrate.

The term “proximal growth” as used herein refers to the growth of bacteria in the electrolyte solution which are located sufficiently close to the coating-electrolyte solution interface as to directly contact the coating composition.

The term “stably adhered” as used herein refers to two substances united by a molecular force acting in the area of contact which is of sufficient strength to avoid dissociation under the anticipated conditions.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

Bactericidal Coating/Composition/Electrode Article

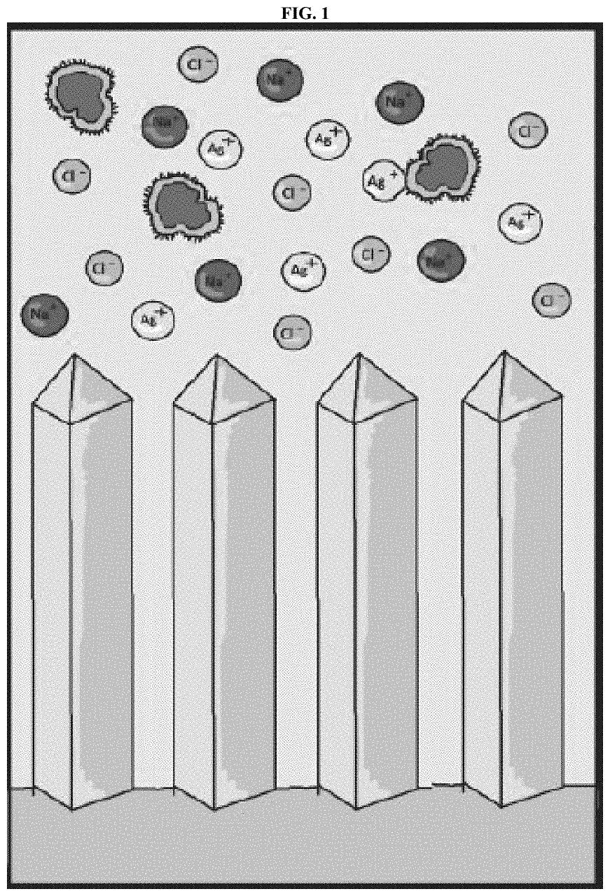

In certain embodiments, the coatings disclosed herein take advantage of the columnar microstructure to provide a mechanism to release bactericidal elements. The current disclosure provides a coating composition comprising a bactericidal element, non-limiting examples including Ag, Cu, Zn, Sn, Au, Ni, and/or Co, wherein the incorporation of the bactericidal element does not disrupt the columnar microstructure and/or does not reduce the efficiency of the underlying electrode, thereby retaining performance while adding bactericidal functionality. Bactericidal ions of the bactericidal element, generated either via passive dissolution or upon application of a potential, are enriched within and near the columnar microstructure. The voids between adjacent columnar microstructures act as a reservoir for bactericidal ions, which is replenished from bactericidal element adjacent to the pores. Ion containing biological solutions, having access to these pores, are susceptible to the activity of the bactericidal ions, whether proximal or distal in solution (FIG. 1). The bactericidal element both reduces the possibility of biofilm formation proximal to the electrode and permits the release of bactericidal ions enabling the killing of distal bacteria.

In one aspect, the coatings described herein are superior to conventional bactericidal coatings comprising small molecule antibiotics. In certain embodiments, loading antibiotic compounds in the correlated pores of such compositions decreases the effectiveness of the coatings for charge exchange. In certain embodiments, the presently described bactericidal ions are selectively released upon application of an electrical potential, whereas no such control is available for the release of antibiotic compounds. In certain embodiments, bactericidal elements are less likely to lead to the development of antibiotic resistance.

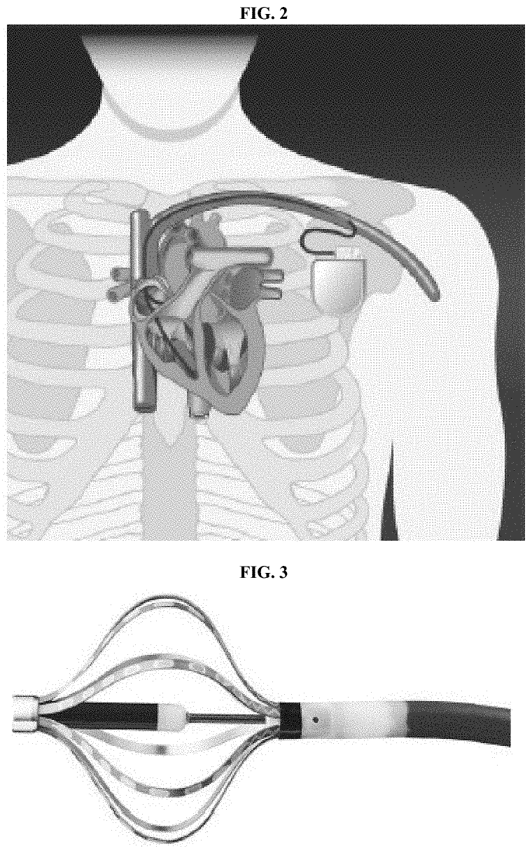

In certain embodiments, the present disclosure is readily applicable to implantable cardiac electrodes, including pacemaker leads (FIG. 2), or the electrodes of other diagnostic tools (FIG. 3). The electrical connection of such devices to the biological system would be enhanced with the presently disclosed coatings. Furthermore, the release of at least a fraction of the bactericidal element would decrease the likelihood of infection during diagnostic testing and/or surgical implantation of the medical device. Thus, the invention allows for reducing infections including, but not limited to, blood, heart, and other implanted device-related infections, and for reducing the number of revision surgeries.

It should be noted that the electrode coating described herein are not limited to implantable devices, but can be applied to any situation wherein proximal and distal bactericidal activity is desired, including, but not limited to, electrolysis cells and aquaria. Furthermore, the present disclosure may be applied to implantable devices that do not require an applied potential in their ordinary function, but rather may tolerate application of a potential.

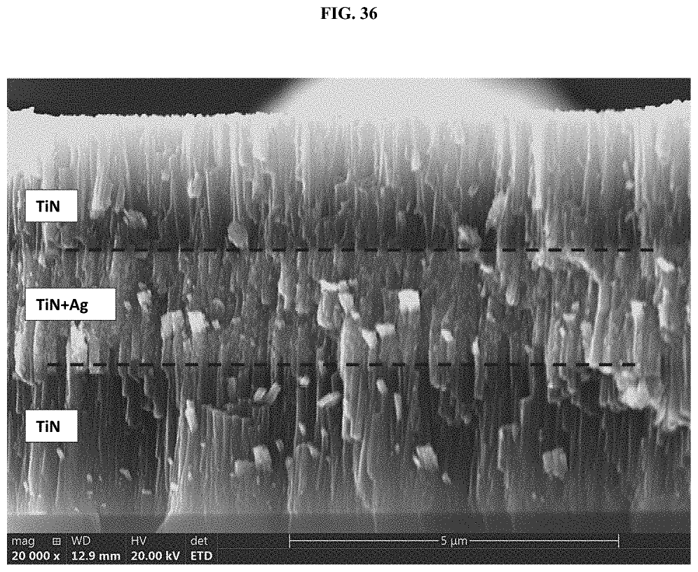

The present disclosure relates in one aspect to coating compositions comprising a bactericidal layer further comprising a bactericidal element and a columnar microstructure. In certain embodiments the bactericidal layer is a multilayer, such as a bilayer, a tri-layer, and so on. It should be noted that, although the instant specification mainly uses bilayer coatings as illustrative non-limiting examples of the multilayer coatings, the instant specification is not limited thereto. Structures such as tri-layer coatings, tetra-layer coatings, and so on are specifically included. For example, an SEM image of an exemplary tri-layer coating is shown in FIG. 36. In other embodiments the bactericidal layer is a composite of the bactericidal element and the columnar microstructure. In certain embodiments the composition is coated on the surface of an object. In certain embodiments the object is selected from the group consisting of intramedullary nails, pins, rods, plates, screws, artificial joints, artificial heart components, prosthetic blood vessels, catheters, stents, wound dressings, surgical stitching fibers, and pharmaceutical depots. In certain embodiments the coating composition effectively reduces or eliminates proximal and distal bacterial growth or adhesion.

The present disclosure relates in another aspect to a coating composition comprising a bactericidal layer further comprising a bactericidal element and columnar microstructure, which is stably adhered to the surface of an electrode. In certain embodiments, the bactericidal layer is a multilayer, such as a bilayer, a tri-layer, and so on. In other embodiments, the bactericidal layer is a composite of the bactericidal element and the columnar microstructure. In certain embodiments, the electrode comprises at least one implantable medical device selected from the group consisting of a pacemaker, cardioverter defibrillator, retinal implant, phrenic nerve stimulator, glucose biosensor, cochlear implant, and an electrical stimulator for pain relief management, Parkinson's disease, and/or epilepsy. In certain embodiments, application of an electric potential to the coating composition releases bactericidal ions. In certain embodiments, the bactericidal ions effectively reduce or eliminate proximal and distal bacterial growth or adhesion.

### EXAMPLES

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Bilayer Coatings

In Examples 1-1 and 1-2, bilayer coatings that are formed by sequentially coating substrates with a bactericidal layer and a columnar microstructure layer. (It should be noted that, although the instant specification mainly uses bilayer coatings as illustrative non-limiting examples of the multilayer coatings, the instant specification is not limited thereto. Structures such as tri-layer coatings, tetra-layer coatings, and so on are specifically included. For example, an SEM image of an exemplary tri-layer coating is shown in FIG. 36.)

### Example 1-1: Ag/TiN Bilayer Coating

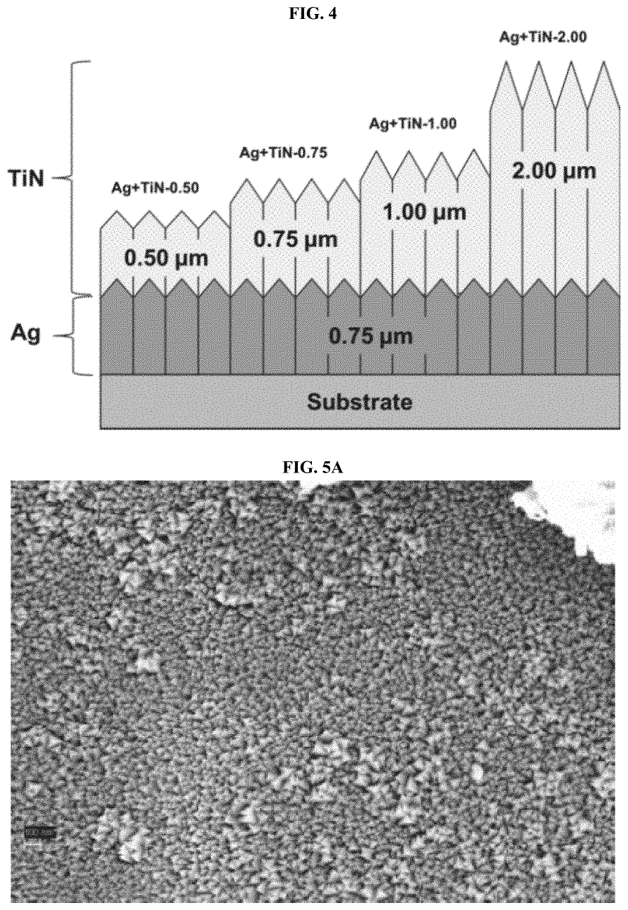

Bilayer coatings of Ag and TiN have been synthesized using DC magnetron sputtering for the Ag layer and DC magnetron reactive sputtering for the TiN layer to arrive at sequentially sputtered coatings (FIG. 4). The thickness of coatings was measured by stylus profilometry on an Ambios XP-2. In certain embodiments, a thickness of 0.75 μm was obtained for the Ag layer. In certain embodiments, a thickness of 0.50 μm, 0.75 μm, 1.00 μm, and 2.00 μm was obtained for the TiN layer. However, coatings with thicknesses other than those described herein may also be achieved utilizing the synthetic method described herein.

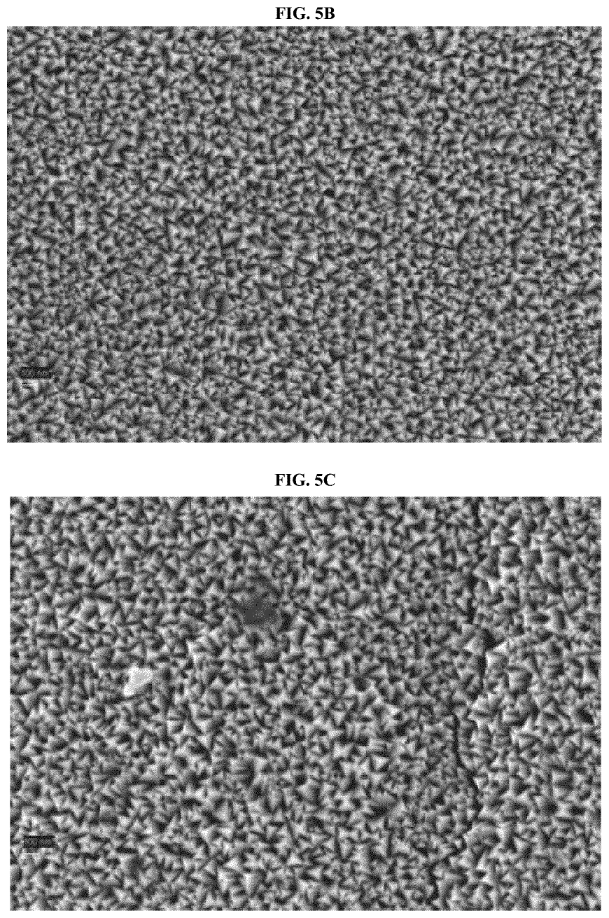

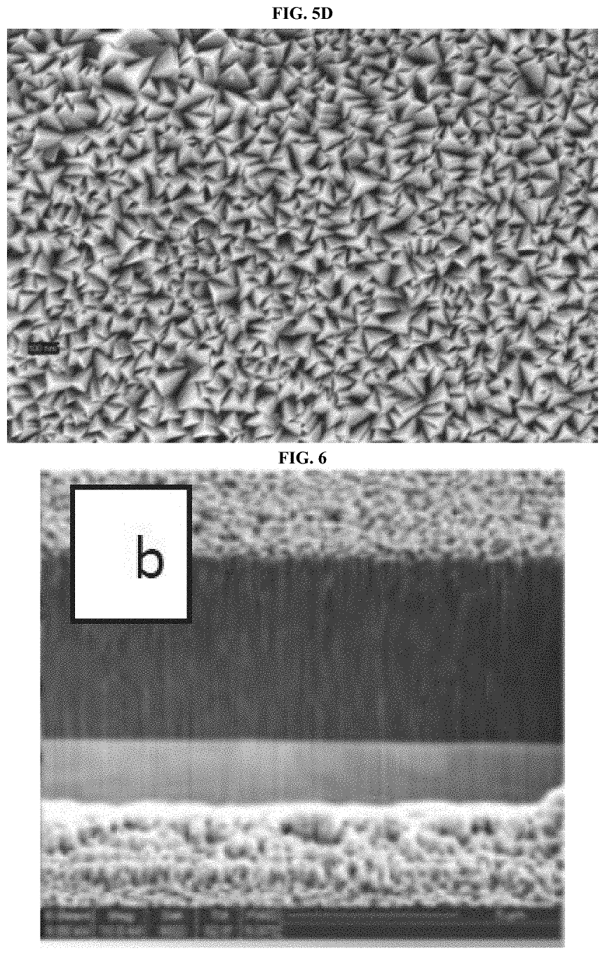

Scanning electron microscopy (SEM) was performed on representative deposition samples demonstrating the “cube-corner” microstructure characteristic of columnar growth at the coating surface (FIGS. 5A-5D). The basal Ag layer does not impact the columnar growth of the coatings. However, grain size appears positively correlated with TiN layer thickness, wherein increasing thickness of the TiN layer results in an increased observed grain size. A SEM image of an ion-milled cross section shows portions of the correlated pores (FIG. 6). The SEM image demonstrates that while the pores do not run in a perfect vertical line, a pathway is nevertheless created whereby the silver layer may be accessed by a solution when the coating is submerged.

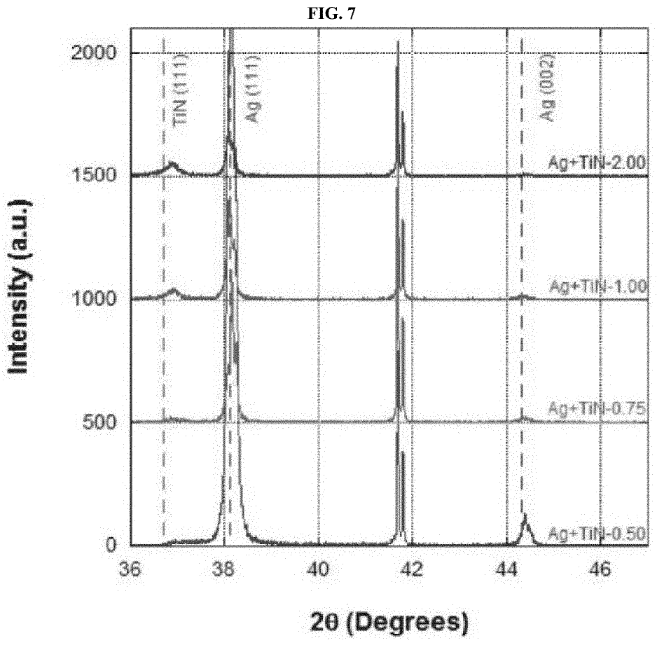

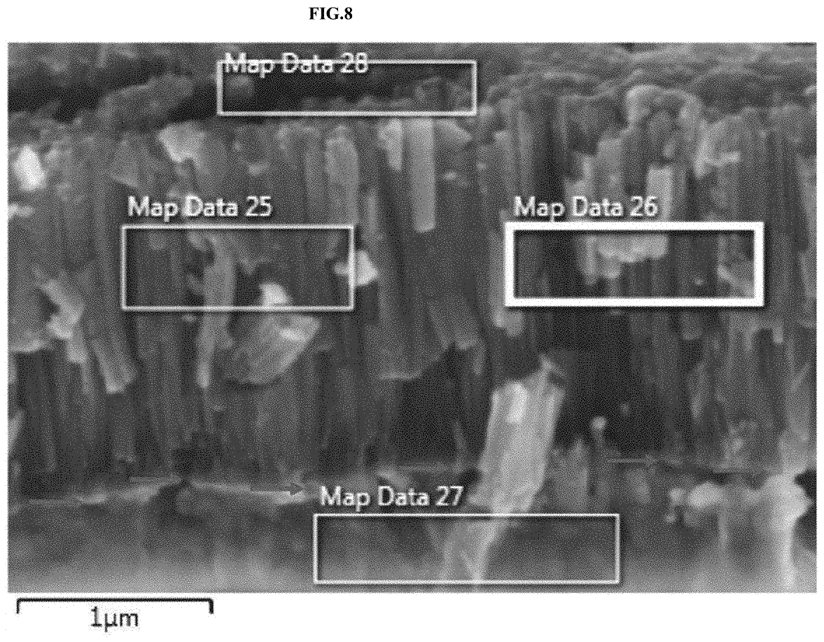

X-ray diffraction (XRD) was used to confirm the discrete nature of the Ag and TiN layers (FIG. 7). The peaks attributed to Ag are greater in intensity as compared to the TiN peaks due to the greater mass and x-ray scattering cross-section of Ag. The only TiN peak observed is the (111) peak, indicating that the coatings are highly textured, which is consistent with microstructural observations. As the thickness of the TiN layer increases, the TiN (111) peak increases in size while the Ag (111) peak decreases. This observation is consistent with the propensity of thicker coatings to attenuate x-rays that reach the silver layer and return through the top layer after diffraction. The sharp peaks observed at 41.6 degrees correspond to the Al₂O₃ single crystal substrate upon which the Ag layer is coated.

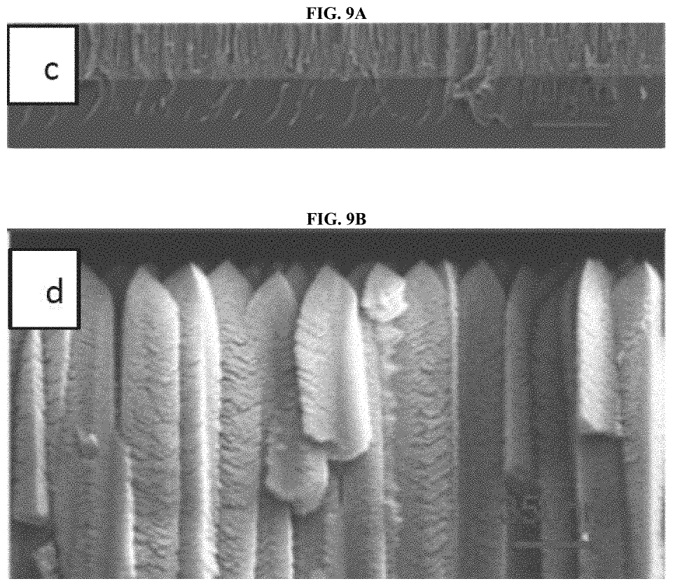

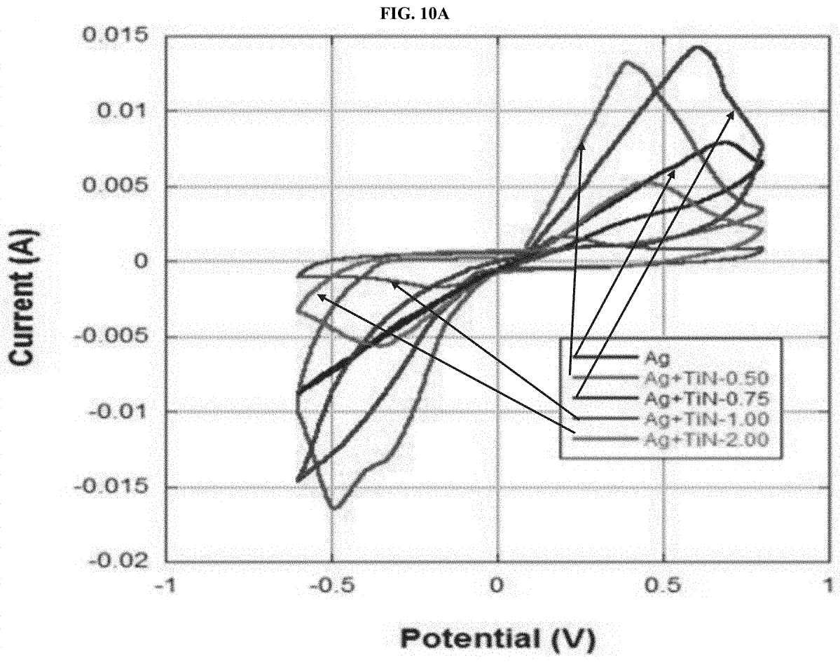

The Ag/TiN bilayer coating compositions were fractured after cooling in liquid N₂ to further confirm the discrete nature of the Ag and TiN layers. The columnar microstructure of the TiN, and the columnar fragments of TiN resulting from the fracture, are clearly observed by SEM (FIG. 8). The composition of the fractured coating was examined by energy dispersive spectroscopy (EDS) (Table 1). A 77% Ag composition was found below the Ag/TiN interface (Ag layer). Similarly, Ag was detected in small quantities above the Ag/TiN interface (TiN layer). The nitrogen content of nearly 20% observed in the Ag layer is likely a result of secondary excitations, wherein the Ag x-ray penetrating above the bilayer interface causes excitation of the low-energy nitrogen x-rays. Comparison with the SEM images of fractured TiN coatings without an added bactericidal element demonstrate that the microstructure is not significantly altered as a result of the added bactericidal element (FIGS. 9A-9B).

TABLE 1
Energy dispersive spectroscopy (EDS) of Ag/TiN
bilayer coating

| Rectangle #^{a} | Ti atomic % | N atomic % | Ag atomic % |
|---|---|---|---|
| 25 | 52.2 | 47.3 | 0.5 |
| 26 | 52.8 | 46.7 | 0.5 |
| 27 | 3.4 | 19.9 | 76.7 |
| 28 | 54.4 | 44.5 | 1.1 |

^{a}Rectangle numbers refer to sections of Ag/TiN coating demarcated in FIG. 8.

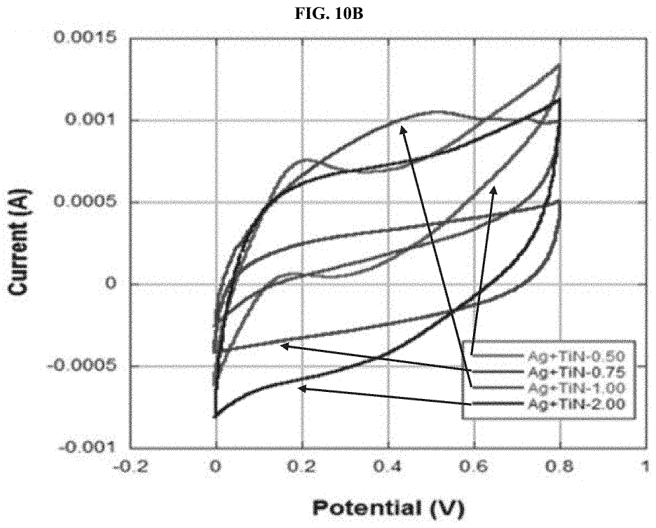

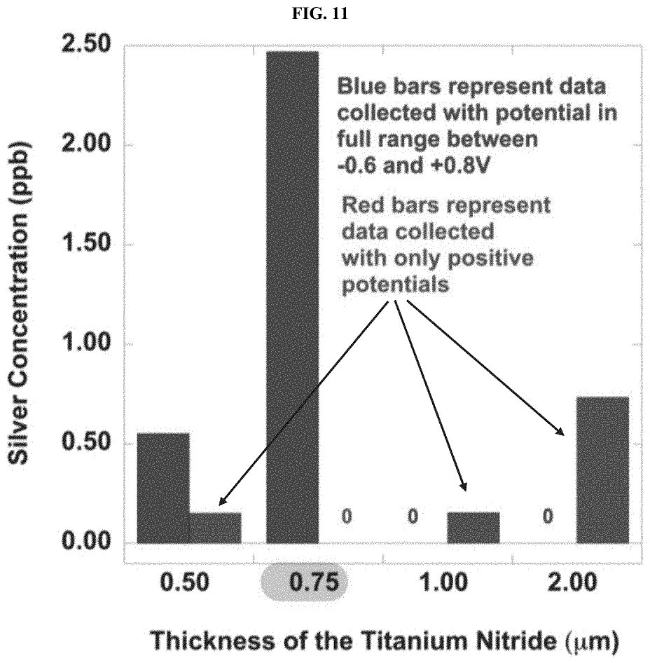

Electrochemical measurements were performed on Ag/TiN bilayer coatings utilizing an electrochemical cell comprising a working electrode (Ag/TiN coating), a counter electrode (platinum coil), and an Ag/AgCl reference electrode. Cyclic voltammetry (CV) was performed with an applied electrical potential difference between the counter and working electrodes, wherein the resulting electrical current was measured (FIGS. 10A-10B). Due to the water content of the electrolyte in the presence of the working electrode material, the range of CV measurements was limited to a range of −0.6 V to +0.8 V. Peaks corresponding to oxidation were observed in CV experiments in which Ag/TiN coatings were evaluated. Coatings comprising TiN without Ag were also evaluated and no oxidation peak was observed. Thus, the observed oxidation peaks are a result of the presence of the Ag, and accordingly, the presence of Ag ions in the electrolyte solution was anticipated. The Ag ion content of each of the electrolyte solutions was determined by inductively coupled plasma-mass spectrometry (ICP-MS) (FIG. 11). No Ag ions above background were observed in the electrolyte solution for CV experiments with applied potential comprising the full water range when Ag/TiN bilayer coatings with a TiN layer thickness of 1.00 μm or greater were evaluated. However, some Ag ions were detected in these thicker Ag/Ti bilayer (≥1.0 μm TiN) coatings when the CV conditions were limited to only positive potentials.

The Ag/TiN coatings with 0.50 μm and 0.75 μm TiN layer thicknesses resulted in the highest oxidation potentials, indicating a greater extent of Ag oxidation. A higher observed Ag ion concentration in the electrolyte after an applied potential is indicative of more facile access of the Ag layer by the electrolyte solution. Additionally, the SEM cross-section of the Ag/TiN coating shows a dense layer near the bottom surface of the coating. Thus, without wishing to be bound by theory, the higher measured Ag-ion concentration observed in the electrolyte solution of the Ag/TiN coating (0.75 μm thick TiN layer), as compared to the 0.50 μm thick TiN layer sample, indicates that the electrolyte has easier access to the Ag layer, and accordingly, the dense interfacial layer of the 0.75 μm thick TiN layer sample has more defects. This result was unexpected, as both layer thicknesses are less than the estimated 1 μm thick dense layer observed in thicker coated samples.

The results of the ICP-MS analysis of the electrolyte solutions indicated that a positive potential does yield silver ions in solution in all but one case. In instances in which the potential is reversed, such that re-deposition of the Ag ions onto the coating can occur, thicker coatings do not yield any measurable ion content in the solution. Without wishing to be limited by any theory, this phenomenon is likely a consequence of the natural diffusion lengths of the ion in solution within the voids between columns. On the basis of this rationale, the diffusion speed of the ions is less than 0.125 μm/s, as the ions do not emerge after a thickness of 1 μm is reached.

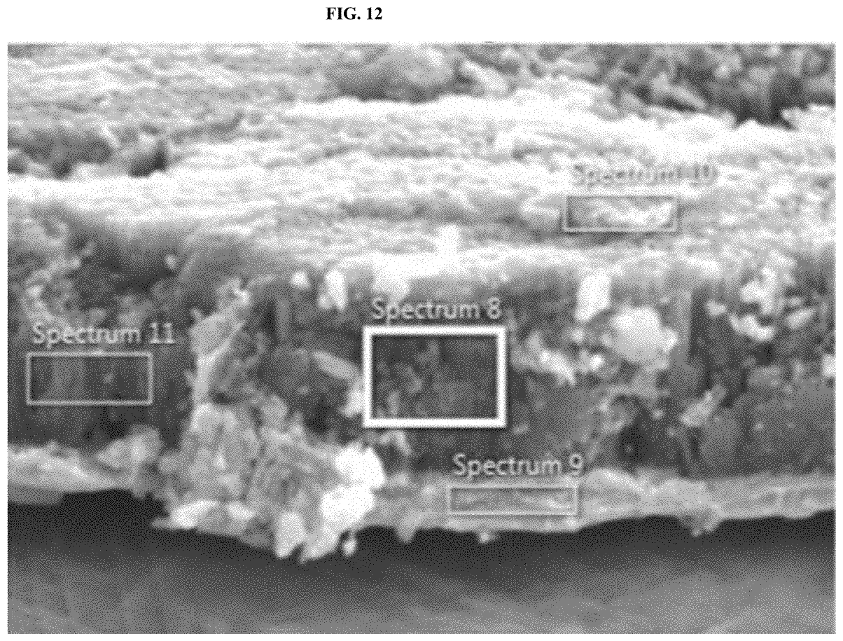

Re-deposition of Ag ions from the electrolyte solution to the columnar TiN surface was observed in a SEM image of a fractured Ag/TiN sample after electrochemical cycling (FIG. 12). Further, the composition of the fractured coating was measured using EDS, demonstrating that the Ag content is uniformly greater in the TiN layer after electrochemical cycling than before (Table 2). These results indicate that the electrolyte solutions access the full thickness of the coatings through the extended columnar pores between the columnar pillars, resulting in oxidation and elution of the Ag ions.

TABLE 2
Energy dispersive spectroscopy (EDS) of fractured Ag/TiN
bilayer coating after electrochemical cycling

| Rectangle #^{a} | Ti atomic % | N atomic % | Ag atomic % |
|---|---|---|---|
| 8 | 51.3 | 47.7 | 1 |
| 9 | 3.4 | 0 | 96.6 |
| 10 | 28.3 | 62.2 | 9.5 |
| 11 | 47 | 51.6 | 1.4 |

^{a}Rectangle numbers refer to sections of Ag/TiN coating demarcated in FIG. 12.

### Example 1-2: Cu/TiN Bilayer Coating

In a manner analogous to the preparation of Ag/TiN bilayer coatings, Cu/TiN bilayer coatings have been synthesized using DC magnetron sputtering for the Cu layer and DC reactive magnetron sputtering for the TiN layer to arrive at sequentially sputtered coatings. The thickness of coatings was measured by stylus profilometry on an Ambios XP-2. In certain embodiments, a thickness of 0.75 μm was obtained for the Cu layer. In certain embodiments, a thickness of 0.50 μm, 0.80 μm, 1.20 μm, and 1.60 μm was obtained for the TiN layer.

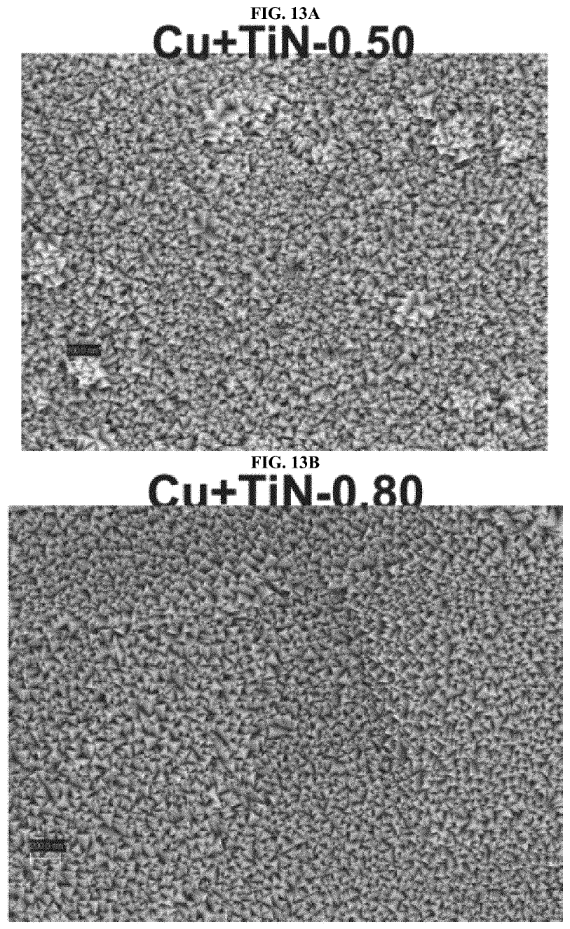

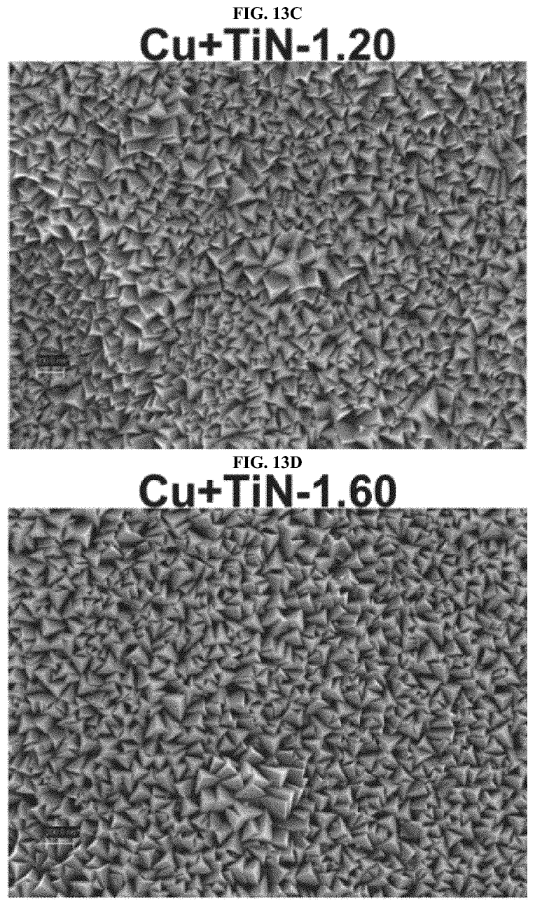

Scanning electron microscopy (SEM) was performed on representative deposited samples, which similarly demonstrate characteristic columnar growth at the coating surface (FIGS. 13A-13D). As observed for Ag/TiN bilayer coatings, the basal Cu layer does not impact the columnar growth of the coatings, and grain size appears positively correlated with TiN layer thickness.

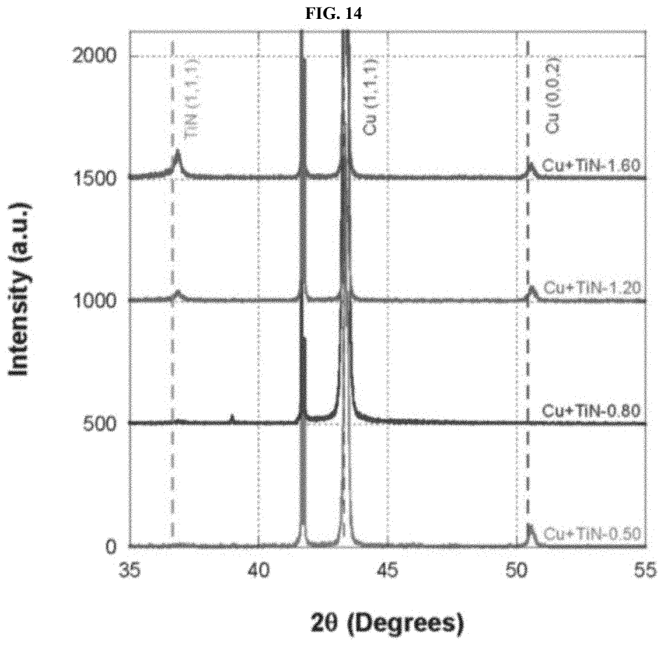

The Cu/TiN bilayer coating was subjected to XRD analysis (FIG. 14). In this case, a clear Cu XRD peak resulting from the pure Cu basal layer is observed. As observed with the Ag/TiN bilayer coating, as the thickness of the TiN layer increases, the TiN (111) peak also increases.

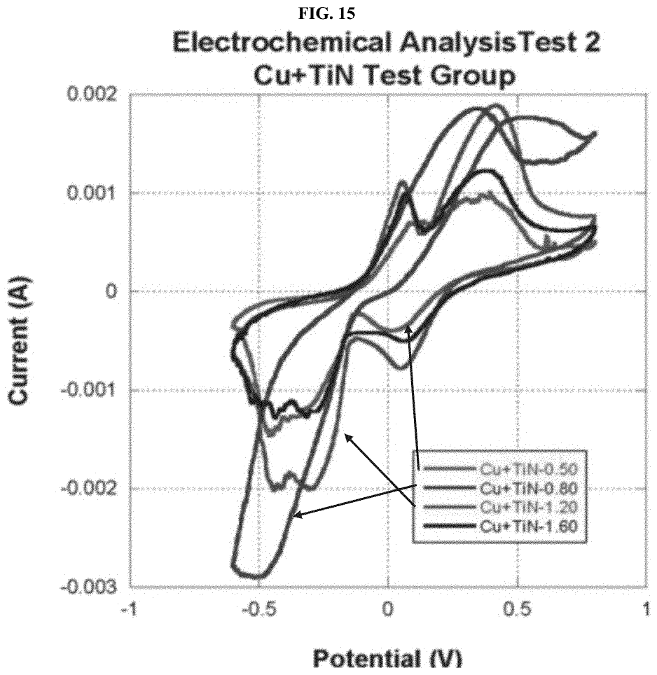

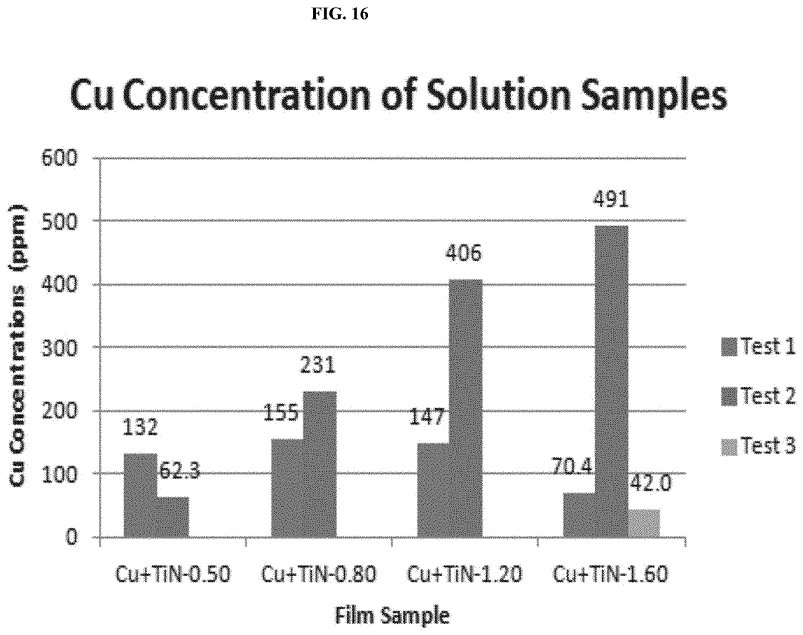

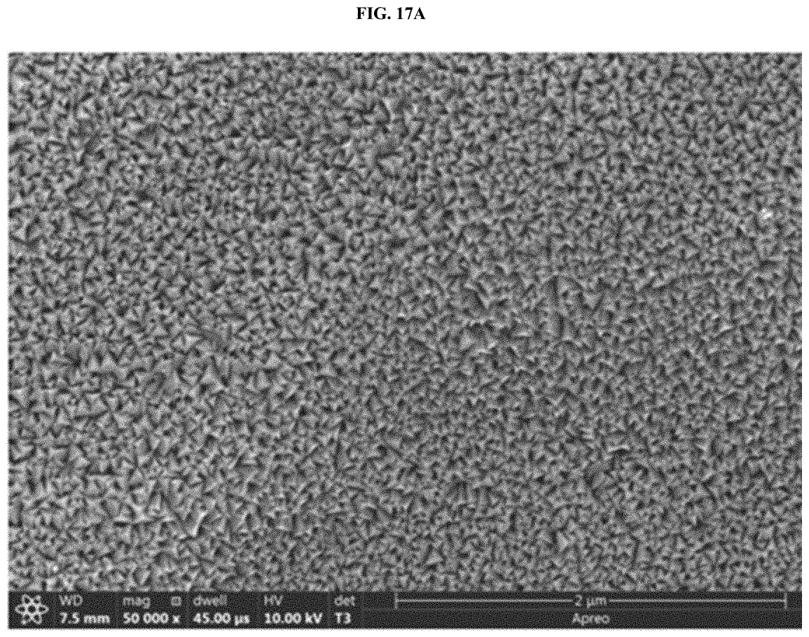

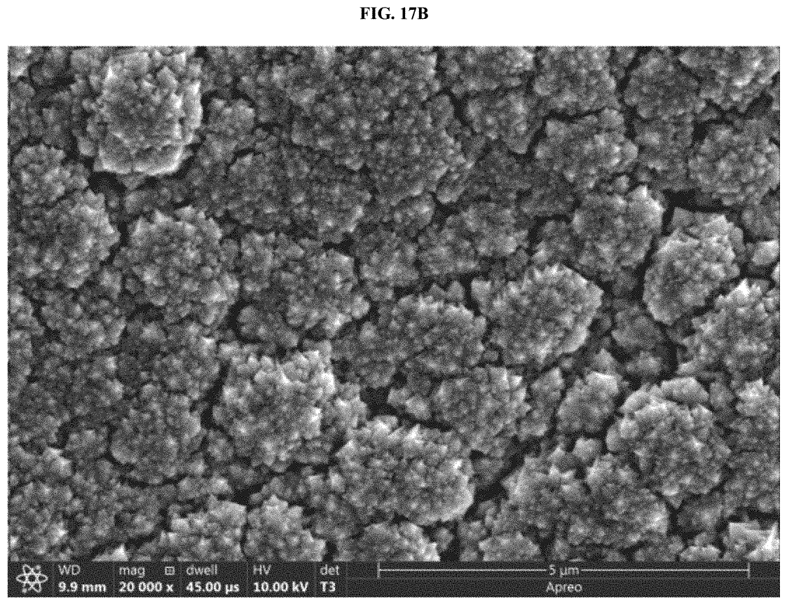

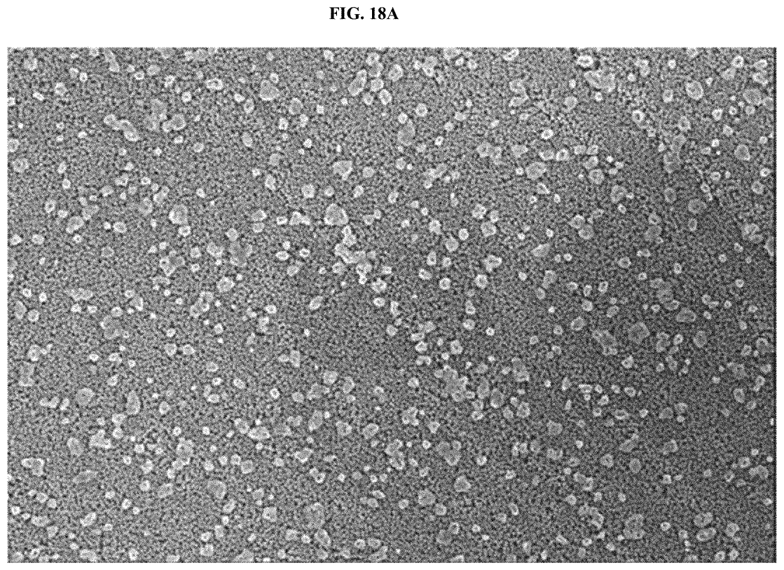

Electrochemical measurements were performed on Cu/TiN bilayer coatings utilizing an electrochemical cell as otherwise previously described (FIG. 15). The Cu ion content of each of the electrolyte solutions was determined by inductively coupled plasma-mass spectrometry (ICP-MS) (FIG. 16). The electrical current observed from the oxidation of Cu was found to be lower than the current observed with Ag, however, the number of Cu ions found in the electrolyte solution is much higher than for Ag. The increased relative abundance of Cu, as compared to Ag, in the electrolyte solution after electrochemical cycling is likely a result of a higher diffusion rate for Cu through the solution and out of the extended pores.

### Example 2: Composite Coatings

In Example 2 several single layered composite coatings, which were formed by co-depositing a bactericide metal element and a nitride columnar microstructure-forming metal in the presence of N₂, are described. It should be noted that the composite coatings as described herein are not limited to the single layered composite coatings. As shown in FIG. 36, the composite coatings single layer can also be used as the bactericide layer in the multilayer structures.

Composite coatings were synthesized by the co-deposition of either Ti or Zr and either Ag or Cu under partial pressure of N₂. A number of coatings were prepared with varied deposition parameters, comprising variations of Ag power, Ti power, and time (Tables 3-5). The composition percentages provided in Tables 3-5 are with respect to the metals only and ignore the nitrogen content. However, the nitrogen atomic content is approximately equal to the titanium content, corroborating that the base material is TiN for each composition.

TABLE 3
Composite Ag/TiN coating deposition parameters

| | Ag | Ti | | | Thick- | | CSC on |
|---|---|---|---|---|---|---|---|
| Sam- | Power | Power | Ag | Ti | ness | Time | SS (20 |
| ple | (W) | (W) | % | % | (μm) | (min) | cycles) |
| 1 | 5.56 | 135.00 | 49 | 51 | 0.778 | 60 | 50.4 |
| 2 | 0.99 | 135.14 | 10 | 90 | 0.539 | 60 | 13.9 |
| 3 | 2.10 | 135.30 | 31 | 69 | 0.605 | 60 | 21.7 |
| 4 | 1.03 | 179.64 | <0.1 | 100 | 0.895 | 53 | 19.0 |
| 5 | 1.46 | 179.56 | 11 | 89 | 0.969 | 60 | 26.2 |
| 6 | 2.13 | 179.61 | 19 | 81 | 1.650 | 120 | 84.6 |
| 7 | 3.12 | 179.66 | 36 | 64 | 1.840 | 120 | 92.3 |
| 10 | 4.17 | 135.38 | 28 | 72 | 1.580 | 151 | 138 |
| 11 | 2.63 | 180.45 | 17 | 83 | 2.180 | 175 | 167 |

TABLE 4
Additional Composite Ag/TiN coating
deposition parameters

| | Ag | Ti 3″ | Ti 2″ | | | Thick- |
|---|---|---|---|---|---|---|
| Sam- | Power | cathode | cathode | Ag | Ti | ness |
| ple | (W) | Power (W) | Power (W) | % | % | (μm) |
| 12 | 4.88 | 180.12 | 91.04 | 27.00 | 73.00 | 1.16 |
| 13 | 5.07 | 180.16 | 91.80 | 22.00 | 78.00 | 2.43 |
| 14 | 2.66 | 179.70 | 91.40 | 16.00 | 84.00 | 2.21 |
| 15 | 7.48 | 179.87 | 92.20 | 37 | 63 | 2.6 |
| 16 | 2.46 | 179.80 | 91.20 | 9.00 | 91.00 | 1.79 |
| 17 | 4.88 | 179.42 | 91.62 | 20.00 | 80.00 | 1.93 |
| 18 | 2.54 | 179.67 | 91.10 | 15.00 | 85.00 | 3.93 |
| 19 | 2.53 | 180.95 | 91.09 | 15.00 | 85.00 | 2.37 |
| 20 | 2.52 | 180.20 | 92.29 | 13.00 | 87.00 | 7.64 |
| 22 | 4.90 | 179.60 | 92.17 | 24.00 | 76.00 | 4.08 |
| 23 | 3.47 | 180.34 | 91.82 | 18 | 82 | 7.04 |
| 24 | 3.55 | 180.68 | 91.74 | 19 | 83 | 3.7 |

TABLE 5
Composite Cu/TiN coating deposition parameters

| | Cu | Ti-3″ | Ti-2″ | | | Thick- |
|---|---|---|---|---|---|---|
| | Power | cathode | cathode | | | ness |
| Sample | (W) | (W) | (W) | Cu % | Ti % | (μm) |
| 1 | 2.47 | 180 | 110 | 4.89 | 95.11 | 7.93 |
| 2 | 2.49 | 180 | 127 | 5.87 | 94.13 | 0.97 |
| 3 | 5.34 | 180 | 93 | 16.52 | 83.48 | 6.45 |
| 4 | 5.38 | 180 | 93 | 17.92 | 82.08 | 0.76 |

Selected Ag/TiN composite coatings were subjected to SEM (FIGS. 17A-17B). Coatings with a Ag concentration greater than 22% demonstrated a disrupted columnar microstructure (FIG. 17A), while the well-defined cube corners on the surface of coatings with lower Ag concentrations are maintained (FIG. 17B). In certain embodiments, if the bactericidal element is Ag and the columnar microstructure is TiN, the percentage of Ag comprising the bactericidal layer cannot be about 15% or about 19%.

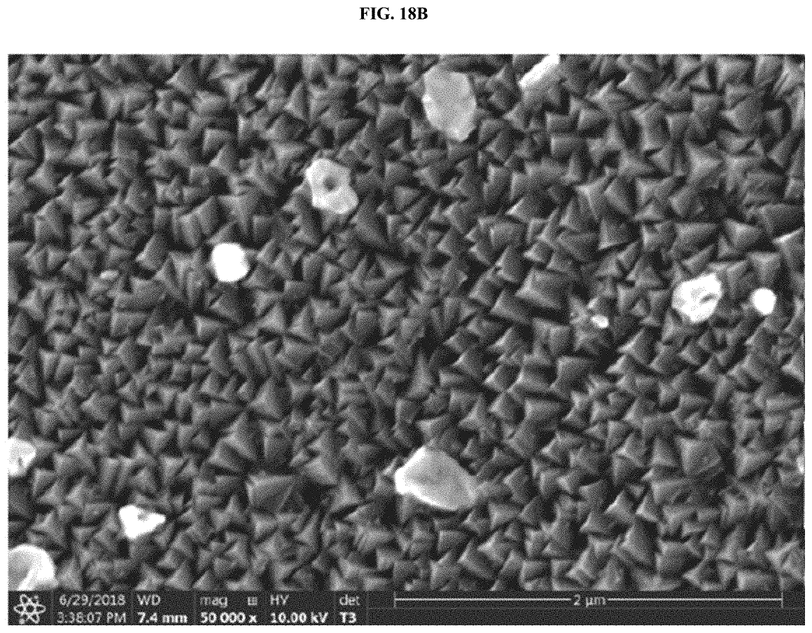

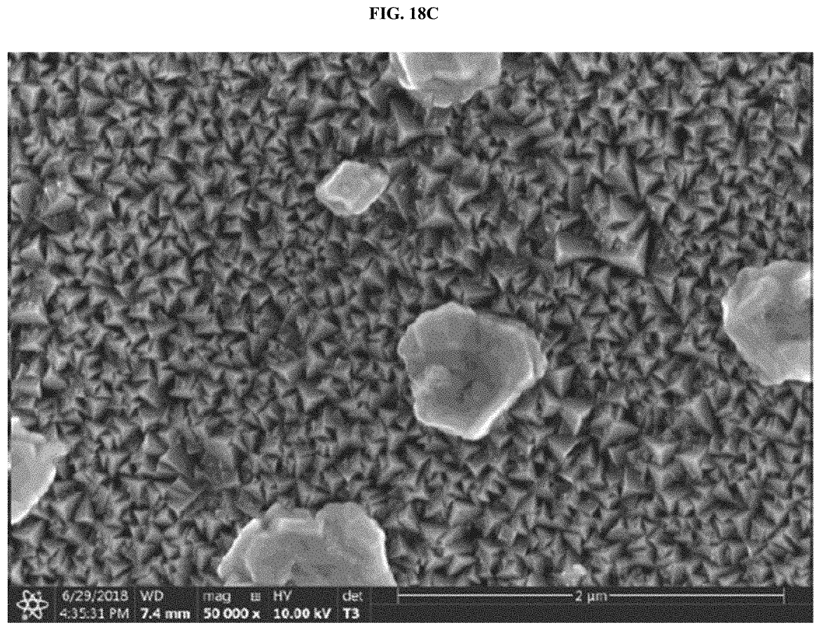

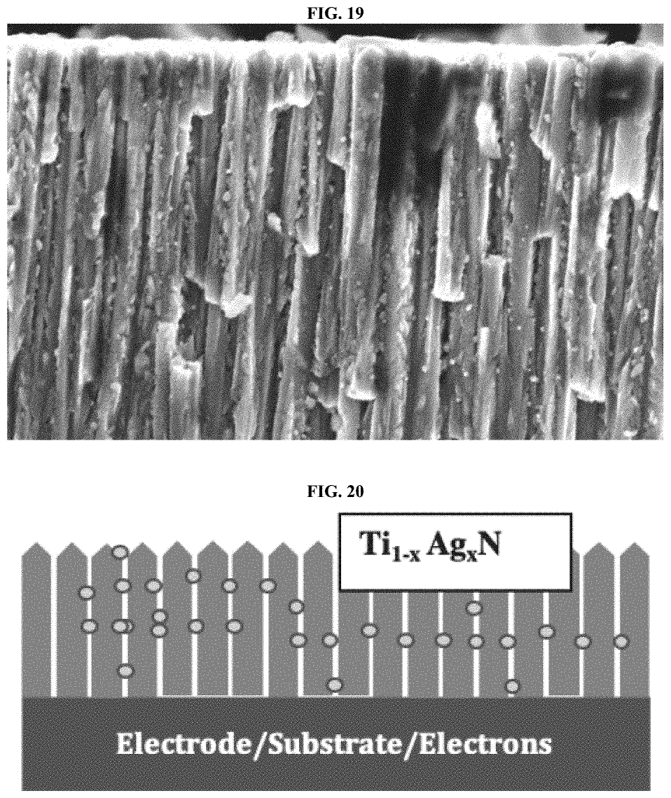

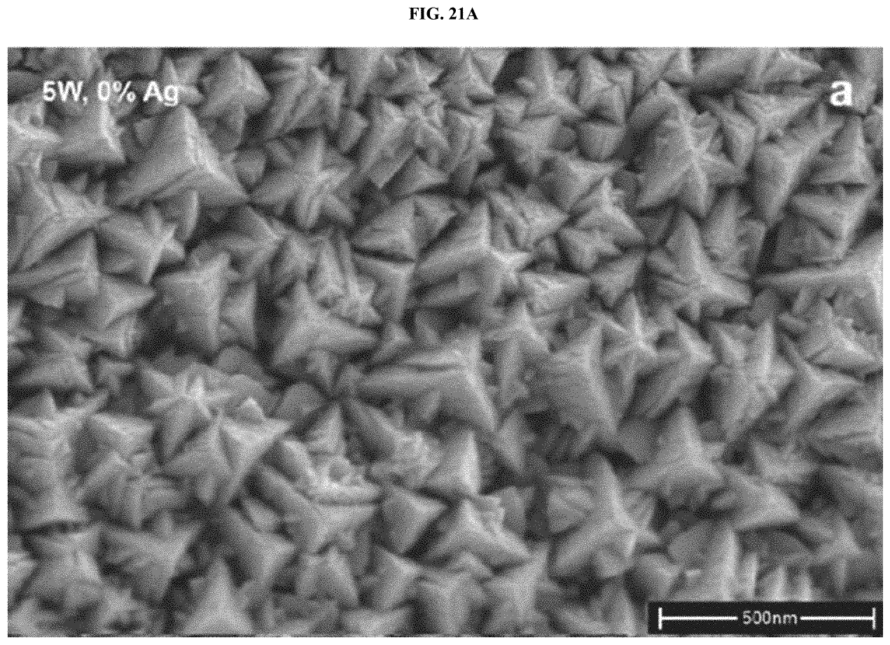

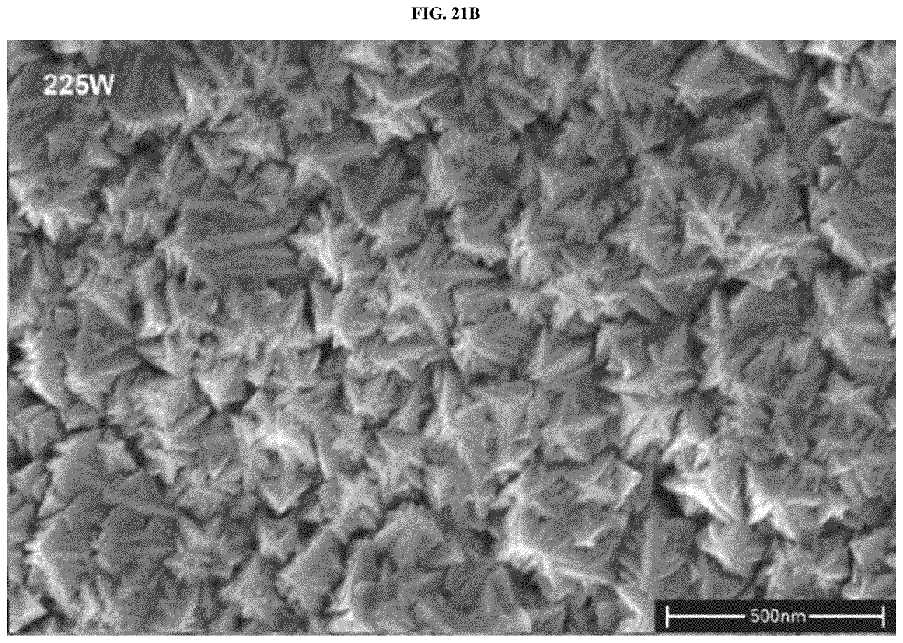

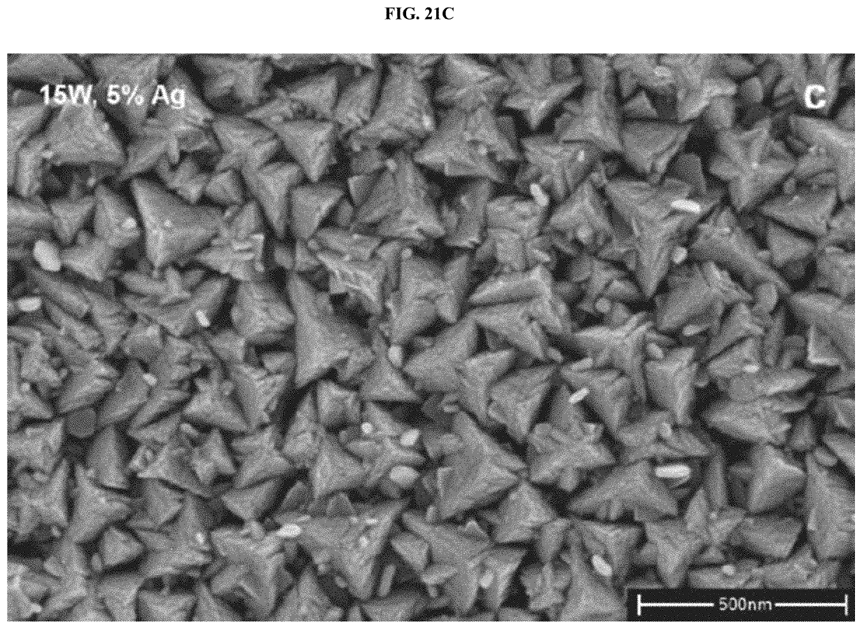

Ag particulates began to appear on the surface of the coatings after several days which were clearly observed by SEM (FIGS. 18A-18C). The relative concentration of Ag in the composite matrix directly influences the size of the Ag particulates observed, wherein a 9% Ag composite resulted in smaller Ag particulates than were observed for the corresponding 20% Ag composite. Additionally, the columns are narrower in the coating with the higher Ag concentration. Nanoparticles of Ag with sizes 50 nm and smaller have been observed on the periphery of columnar microstructures of fractured Ag/TiN composite coatings by SEM (FIG. 19). This phenomenon wherein Ag is expelled from the TiN matrix provides access for bactericidal contact with an electrolyte solution (FIG. 20). Furthermore, surface nanoparticle formation may be beneficial for certain applications, wherein suppression of biofilm formation is desirable.

In contrast to composite Ag/TiN coatings, incorporation of Cu as the bactericidal element in composite TiN coatings does not result in the formation of Cu nanoparticles. The addition of Cu does not influence the pillar structure. Without wishing to be bound by theory, this phenomenon, or lack thereof, is likely the result of a combination of possible copper nitride (CuN) formation, and the relatively small size of the Cu atom as compared to Ag. However, nanoparticle formation is not a requisite characteristic of bactericidal activity.

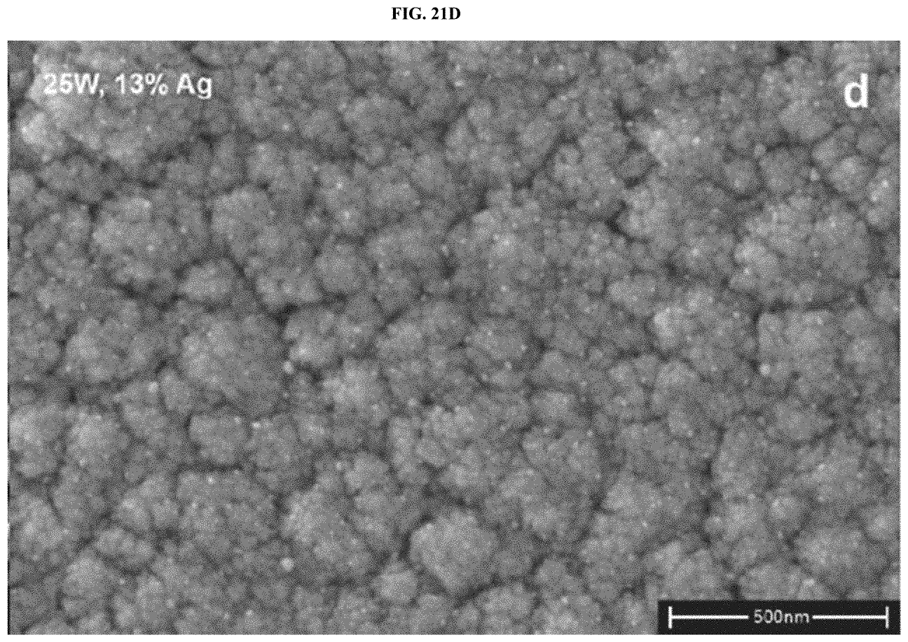

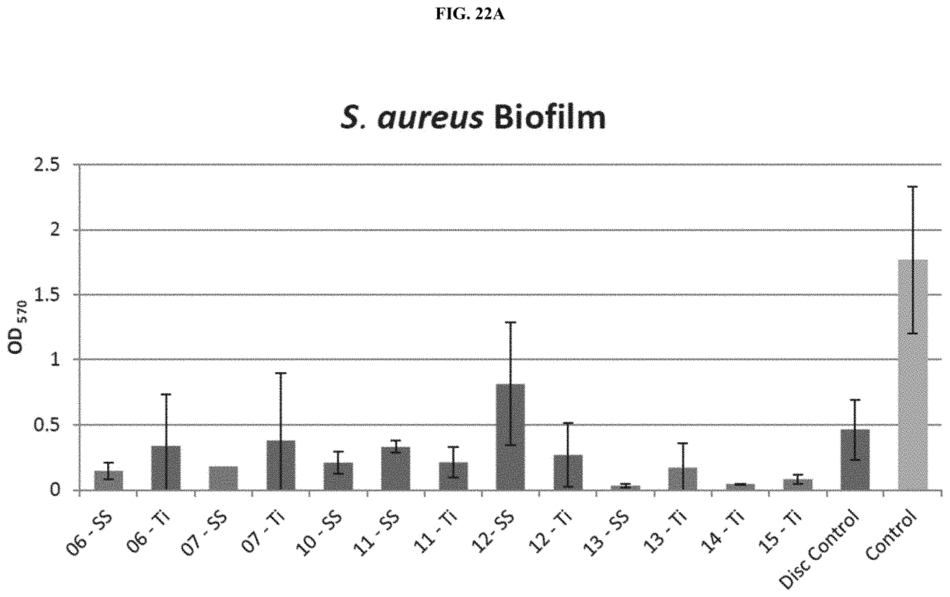

The migration of Ag to the periphery of the columnar microstructures was examined with Ag/ZrN composite coatings. The SEM images of a several Ag/ZrN composite coatings prepared with varied deposition parameters were obtained (FIG. 21A-21D). These images indicate that the best materials, with respect to the presence of “cube-corner” microstructures, are prepared at powers of 135 W, and that the structure changes as the power increases to 225 W. As observed for Ag/TiN composite coatings, after several days Ag nanoparticles are observed for Ag/ZrN composite coatings. However, the Ag particulates observed after expulsion from the ZrN matrix are smaller than observed with Ag/TiN coatings, but display fairly high areal density. Thus, this may represent an approach to control the size and distribution of the bactericidal element particulates.

### Example 3: Microtiter Dish Biofilm Formation Assay

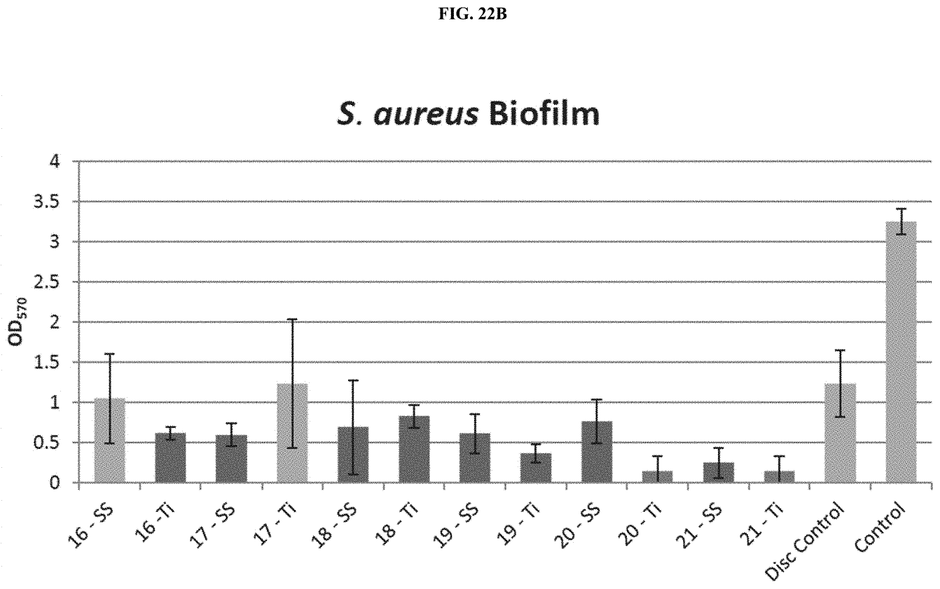

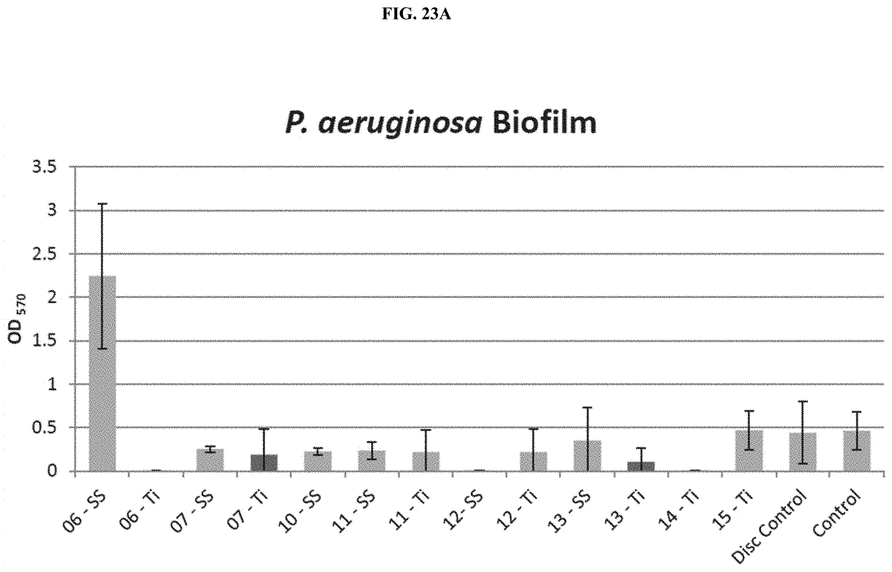

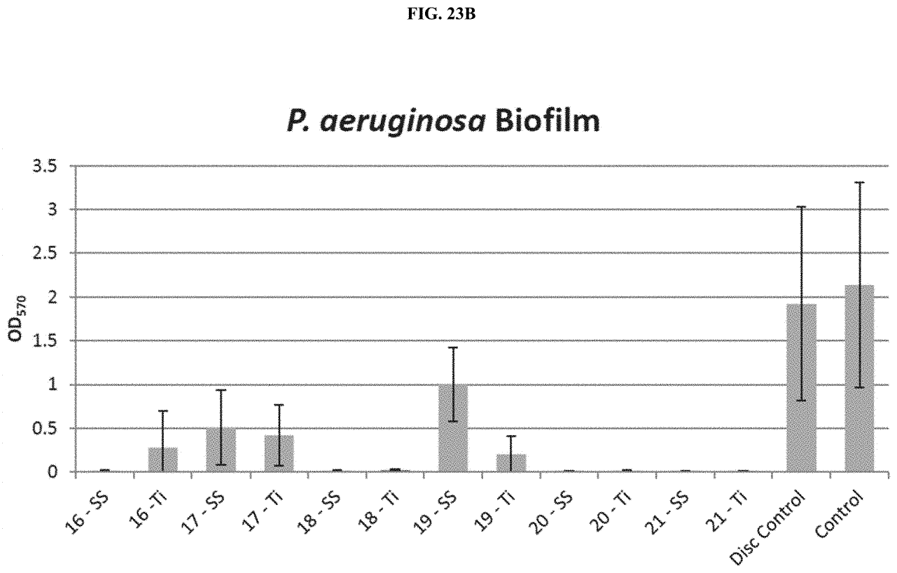

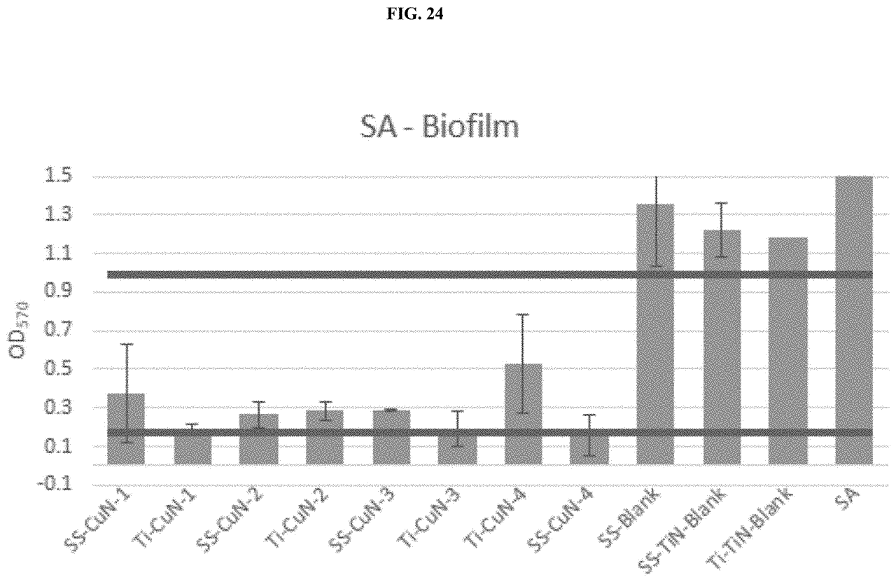

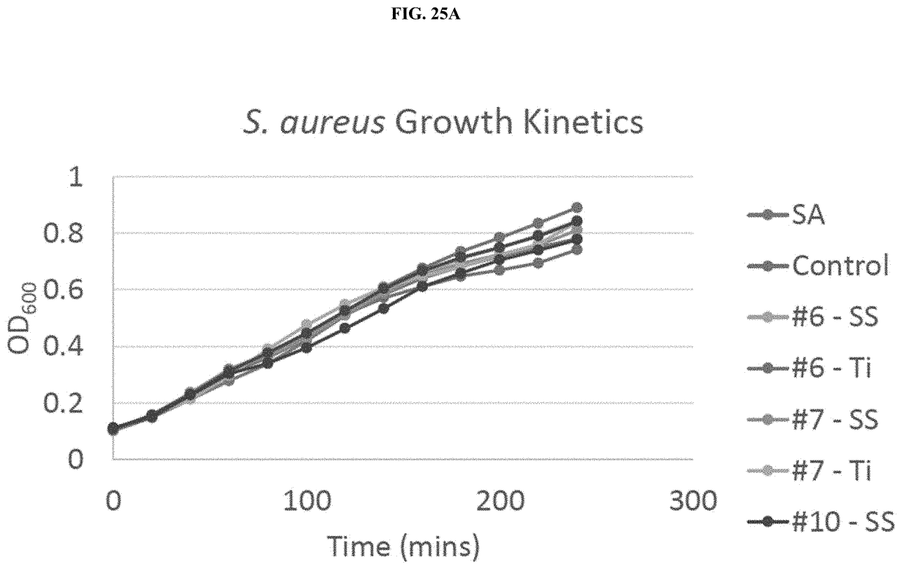

Bactericidal activity of coated metal discs on static biofilm formation of S. aureus and P. aeruginosa was evaluated using the crystal violet staining method. Bactericidal metal discs were added to wells of 96-well microplate before static incubation at 37° C. After incubation, plates were rinsed to remove planktonic cells and adherent cells were quantified by staining with crystal violet and subsequently measuring absorbance at 570 nm. The resultant absorbance values using Ag/TiN coatings were plotted for S. aureus (FIG. 22A-22B) and P. aeruginosa (FIG. 23A-23B), wherein the bactericidal coated metal discs displayed some inhibitory activity against biofilm formation. Similarly, the resultant absorbance values using Cu/TiN coatings were plotted for S. aureus (FIG. 24), demonstrating suppression of biofilm formation in each experiment using Cu/TiN coated materials. Thus, such coatings possess bactericidal activity against biofilm formation without the need for an applied potential.

### Example 4: Growth Kinetics

Each bactericidal coated metal disc was placed in a sterile test tube with 2 mL of Luria-Bertani (LB) media, and the mixture was inoculated with 1 mL of either S. aureus or P. aeruginosa which were grown to an OD₆₀₀ of approximately 0.200. The samples were incubated at 37° C. for 4 hours and the absorbance (600 nm) of each sample was measured at every 20 minutes. After an incubation period of 4 hours, a 1 μL aliquot was removed from each sample and spotted onto a Luria-Bertani agar plate which was further incubated and monitored for colony growth.

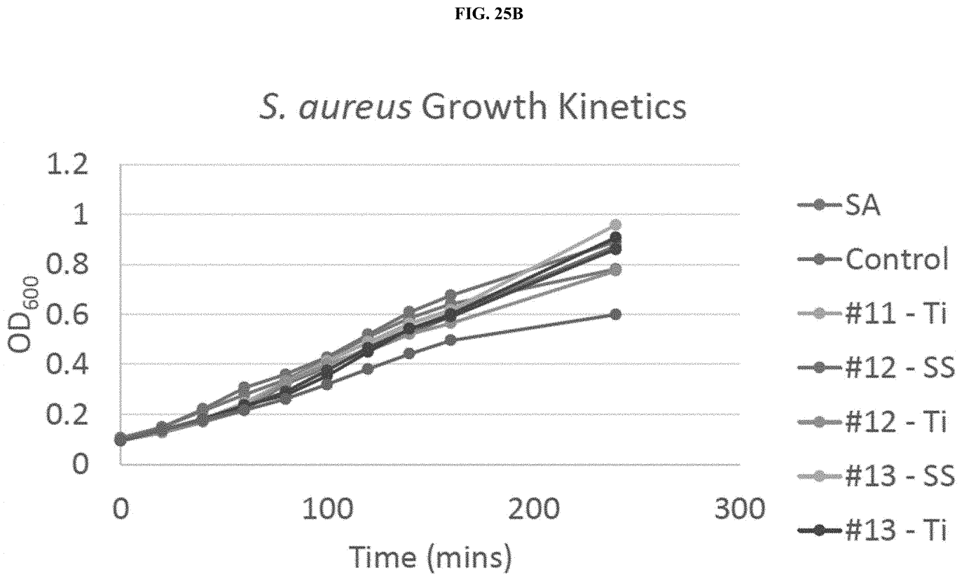

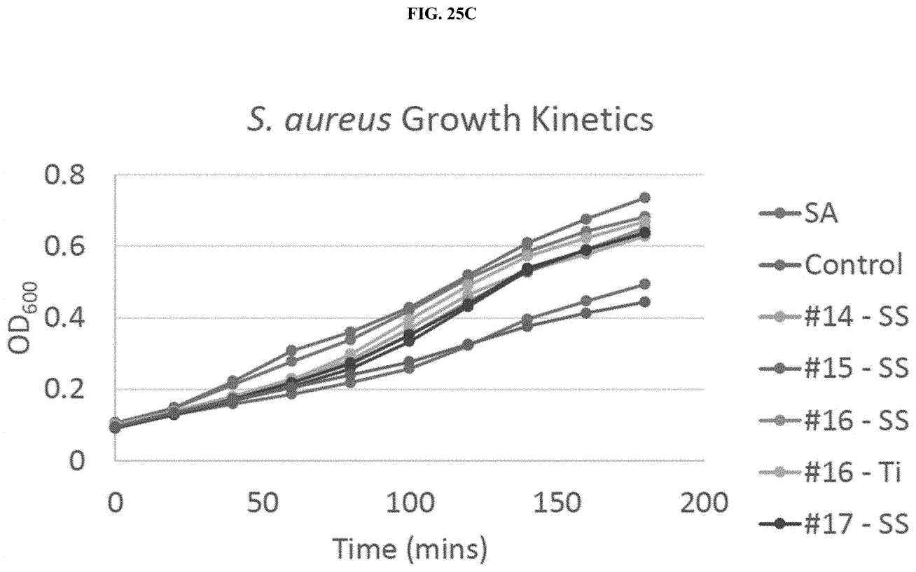

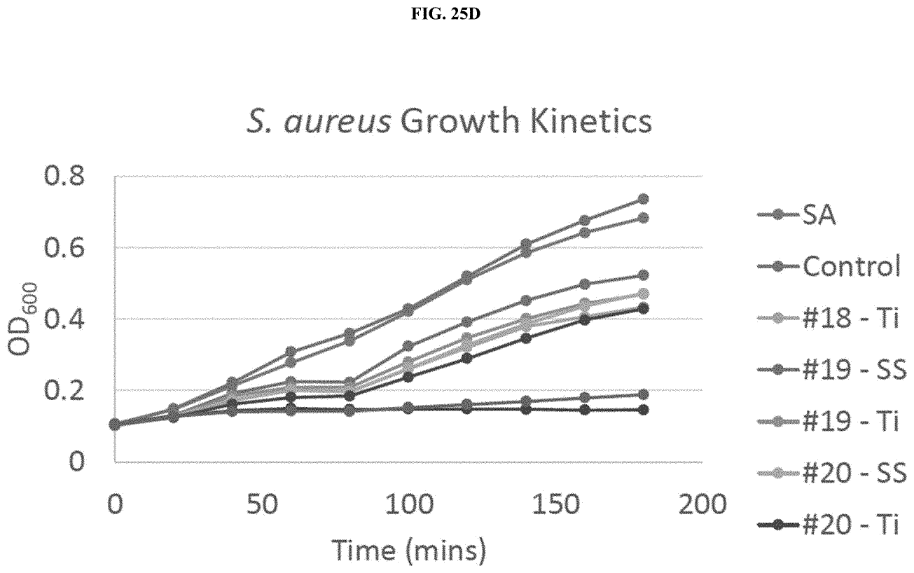

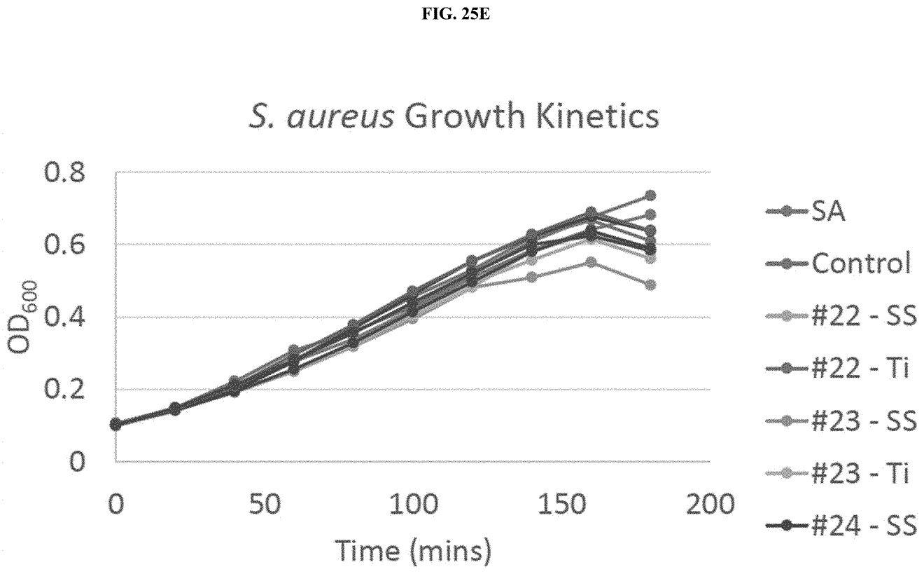

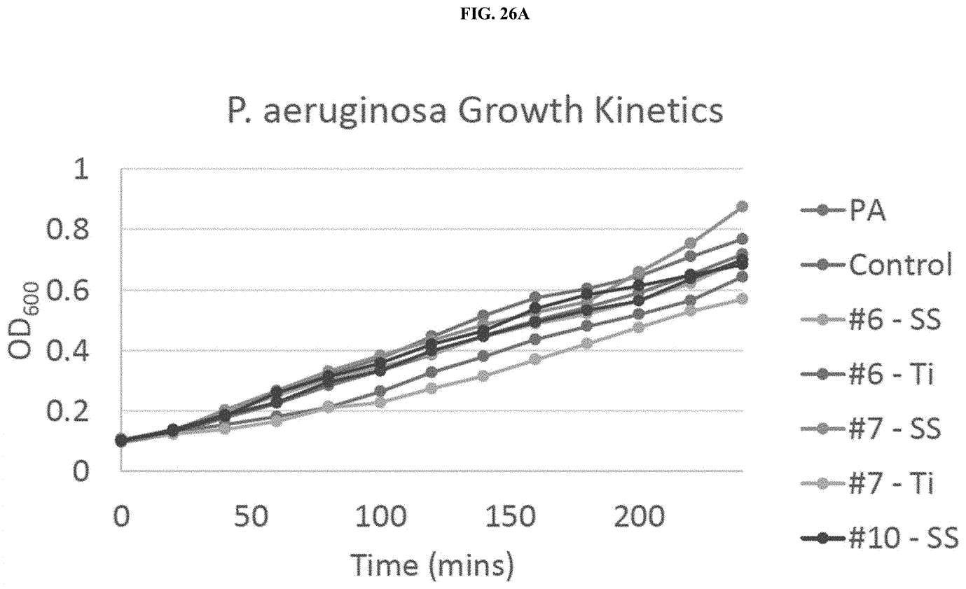

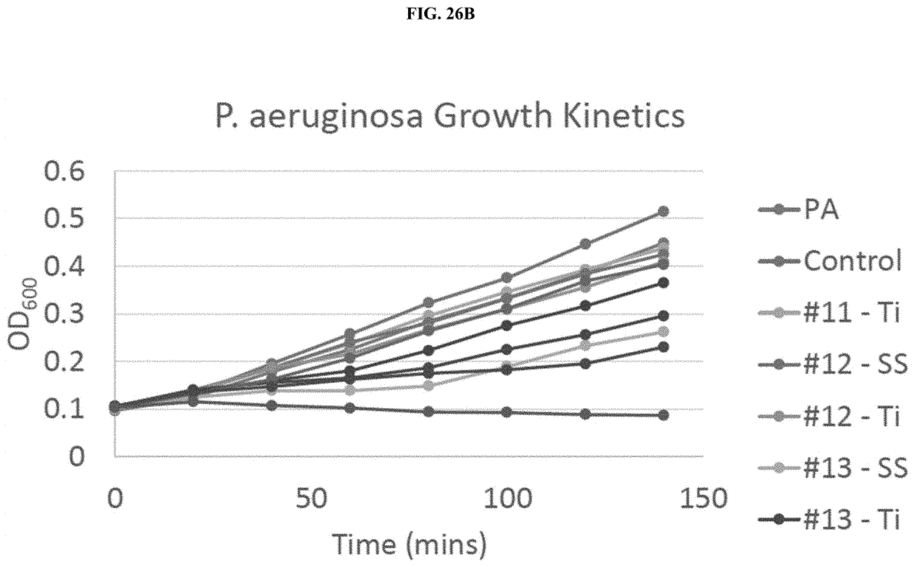

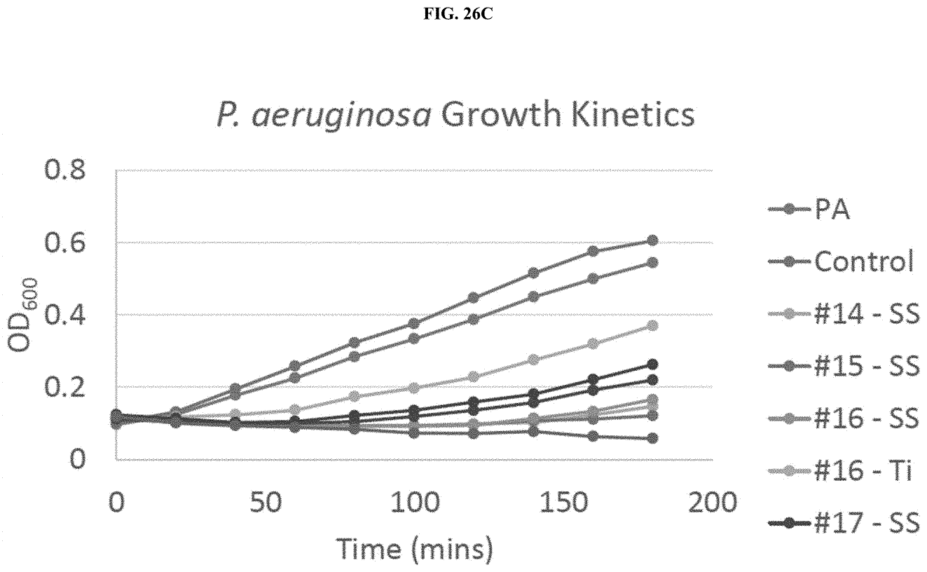

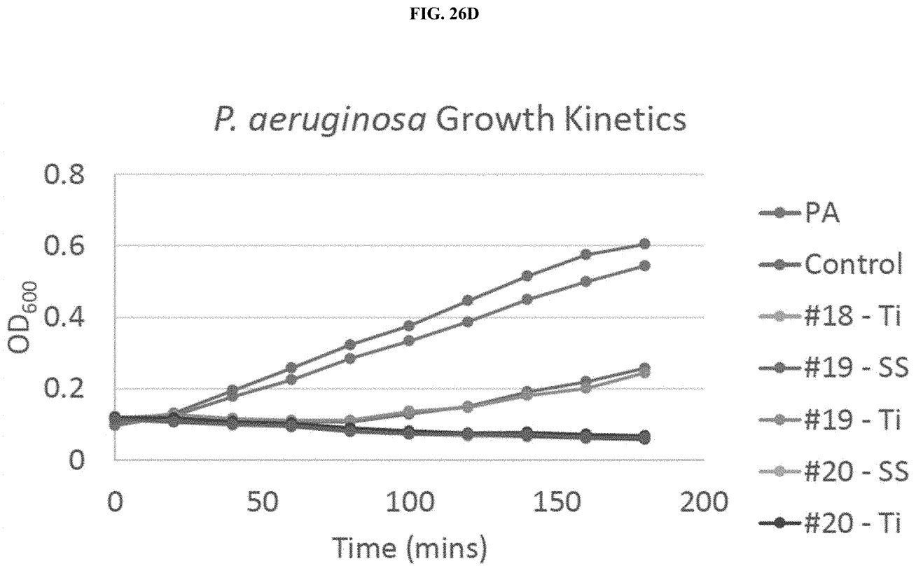

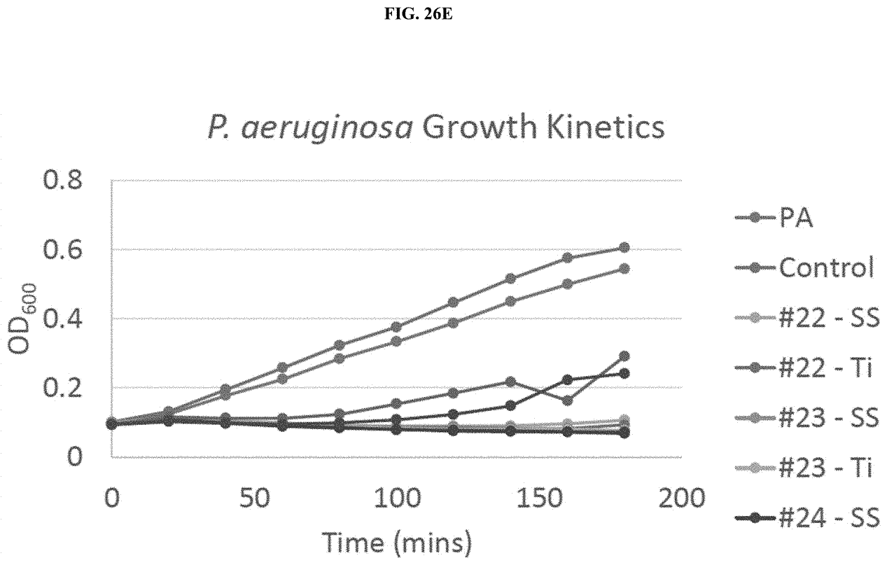

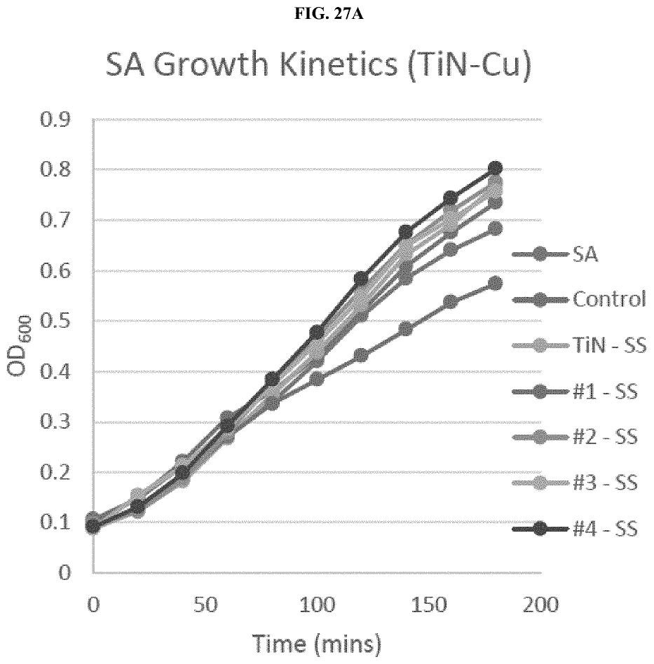

The bactericidal activity of coated discs was quantitatively examined. The results indicate that Ag/TiN coatings with low a low Ag composition do not possess bactericidal activity against distal S. aureus, however Ag/TiN coatings with a high Ag composition possess some bactericidal activity against distal bacteria even in the absence of an applied potential (FIGS. 25A-25E). Similar results were observed with P. aeruginosa, wherein some bactericidal activity against distal bacteria was observed in the absence of an applied potential (FIGS. 26A-26E).

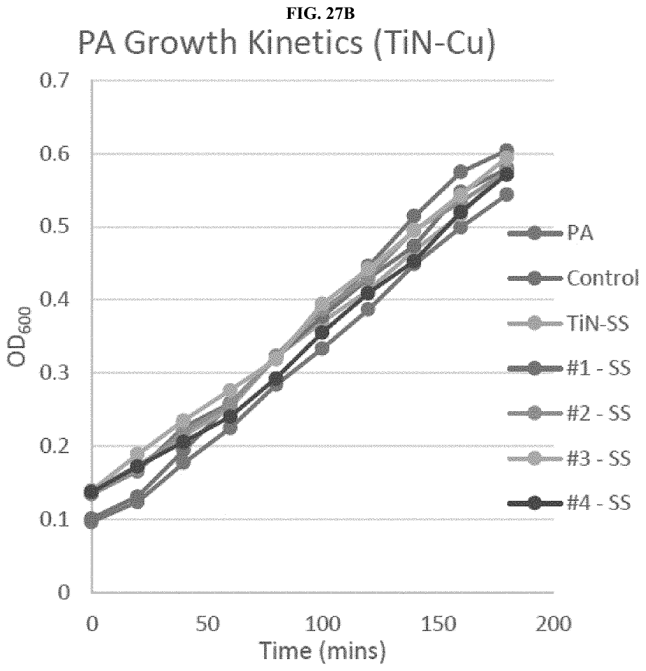

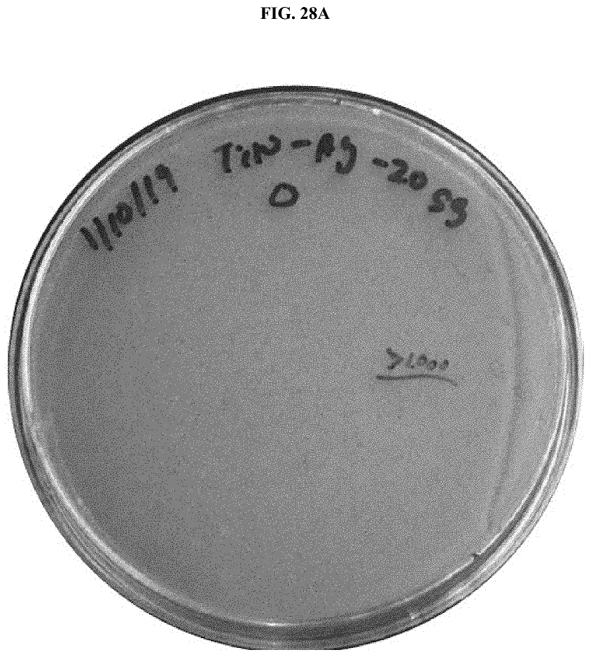

Similar experiments were performed Cu/TiN coated materials (FIG. 27A-27B). Whereas bactericidal activity was observed for Ag/TiN coatings in the absence of an applied potential, no bactericidal activity was observed under the same conditions using Cu/TiN coatings with either S. aureus (FIG. 27A) or P. aeruginosa (FIG. 27B).

### Example 5: Cyclic Voltammetry (CV)

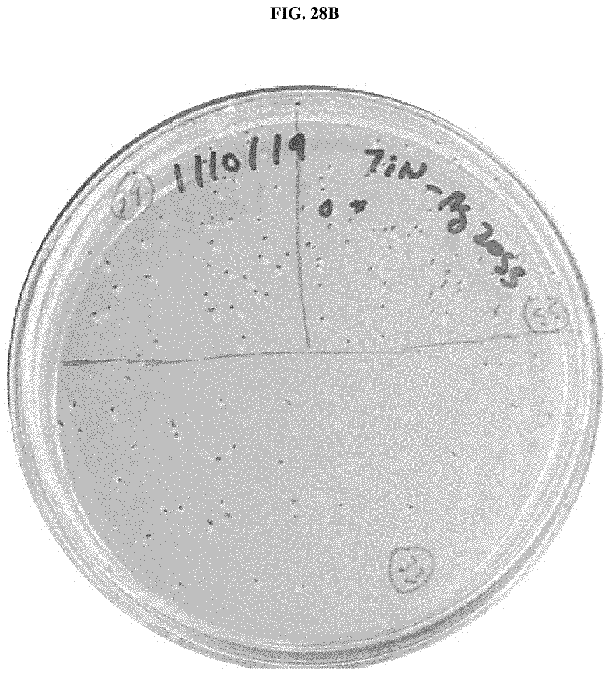

Overnight cultures of P. aeruginosa were diluted 1:200 in LB and grown to an OD₆₀₀ between 0.200 and 0.600 to prepare a stock solution. Utilizing an OD₆₀₀ to CFU conversion factor, the stock solution was diluted to 1×10⁻⁴ CFU/mL in pH 7 PBS buffer, which was used directly in CV experiments (either 20 mL or 1 mL of stock solution total volume). The electrochemical cell used in the CV experiments comprised a Ag/TiN coated stainless steel working electrode (Ag/TiN Sample 20, Table 4), a platinum counter electrode, and a reference electrode. The potential was cycled from 0 V to 0.8 V to 0 V 20 times and the current was measured. Immediately following electrochemical cycling, a 200 μL aliquot was removed and plated on a LB agar plate. Similarly, an aliquot was removed and diluted 1:10 and 200 μL of the diluted sample was plated on a LB agar plate. The remaining solution was incubated at 37° C. and allowed to shake for 30 to 60 minutes, before an aliquot was removed and plated on a LB agar plate. LB agar plates were allowed to incubate at 37° C. with shaking for 12-18 hours. After the incubation period colonies grown on agar plates were counted and compared to the control (FIG. 28A-28B) to afford a quantitative analysis of bactericidal activity the coatings under an applied potential (Table 6). All conditions and procedures were the same for both the 20 mL and 1 mL scale experiments, except that in the 1 mL scale experiment the only aliquot that was plated on LB agar came directly after electrochemical cycling without dilution.

TABLE 6
CFU data from CV experiments after 20 cycles with
Ag/TiN coatings^{a}

| Sample | Time | Initial | Final | % Reduction |
|---|---|---|---|---|
| Volume (mL) | (mins) | CFU/mL | CFU/mL | |
| 20 | 0 | 1E+04 | —^{b} | — |
| 20 | 30 | 1E+04 | 1.48E+02 | 98.53% |
| 20 | 60 | 1E+04 | 0 | 100% |
| 1 | 0 | 1E+04 | 5.23E+02 | 94.78% |

^{a}Ag/TiN Sample 20 (Table 4) was used in these experiments (13% Ag and 87% Ti)
^{b}An accurate CFU/ml value could not be determined due to excessive colony formation

The results of the CV experiments employing bactericidal coatings demonstrate significant distal bactericidal activity against P. aeruginosa, wherein a reduction of 98.53% and 100% were achieved after 30 and 60 minutes, respectively. However, immediately after the electrochemical cycling no significant reduction was observed. These results indicate that the diffusion of eluted ions is slow and that time is required for substantial bactericidal activity when the volume is significant. In experiments in which the total volume was 1 mL, as opposed to 20 mL, a reduction of 94.78% was observed after electrochemical cycling. Thus, longer waiting times or reduced volume lead to substantial, or complete, reduction of the bacterial population.

Similar CV experiments were performed using Cu/TiN coatings under an applied potential to evaluate bactericidal activity against P. aeruginosa with a total volume of 1 mL in all cases. In all cases, even immediately after electrochemical cycling, substantial bactericidal activity was observed, with >50% of reduction of the bacterial population (Table 7). Thus, Cu/TiN coatings also demonstrate significant bactericidal activity under applied potential.

TABLE 7
CFU data from CV experiments after 20 cycles with
Cu/TiN coatings

| | Time | Initial | Final | % |
|---|---|---|---|---|
| Sample | (mins) | CFU/mL | CFU/mL | Reduction |
| TiN Cu (SS) #1^{a} | 0 | 1E+04 | 5.65E+02 | 94.35% |
| TiN Cu (SS) #1^{a} | 0 | 1E+04 | 1.55E+02 | 98.45% |
| TiN Cu (SS) #1^{a} | 30 | 1E+04 | 0 | 100% |
| TiN Cu (SS) #2^{b} | 0 | 1E+04 | 4.98E+03 | 50.02% |
| TiN Cu (SS) #2^{b} | 30 | 1E+04 | 8.0E+01 | 99.2% |

^{a}Cu/TiN Sample 1 (Table 5) was used in these experiments (4.89% Cu and 95.11% Ti);
^{b}Cu/TiN Sample 2 (Table 5) was used in these experiments (5..87% Cu and 94.13% Ti)

### Example 6: Impacts of Bactericide Element on Columnar Microstructures Formations

In non-limiting embodiments, impurities, bactericidal or not, added to the columnar microstructures could reduce the size of or completely block the pores between the columnar microstructures.

Since the bactericidal elements sometimes have small solubilities in aqueous solutions, efficacy of the coatings sometimes requires a potential to be added to the coatings (as would be applied in normal operation of some implantable articles) to oxidize the bactericidal elements, thereby releasing bactericidal ions.

The present study therefore provides results of measurements of the range of impurity (bactericidal) element additions that retain the important columnar microstructure.

Coatings were synthesized using reactive dc-magnetron sputtering. Bactericidal elements were added to the TiN by co-sputtering from an elemental source. The amount of bactericidal element was controlled in the coating by adjusting the power to the source (increased power results in increased deposition rate), or by variation of the distance from the source to the substrate. In the latter case, many different concentrations of bactericidal element could be investigated in a single deposition.

After deposition, the microstructure of the bactericidal element containing coatings was characterized using scanning electron microscopy (SEM). The composition of the coatings was measured using energy dispersive spectroscopy (EDS). X-ray diffractometry was used to investigate the role of the bactericidal element on the crystal texturing in the coating which is strongly correlated with the columnar microstructure.

In certain embodiments, different bactericidal atoms can have differing impact on the TiN microstructure due to the mass of the element and how they sputter among other potential parameters. In what follows, representative data that was used in this analysis will be introduced.

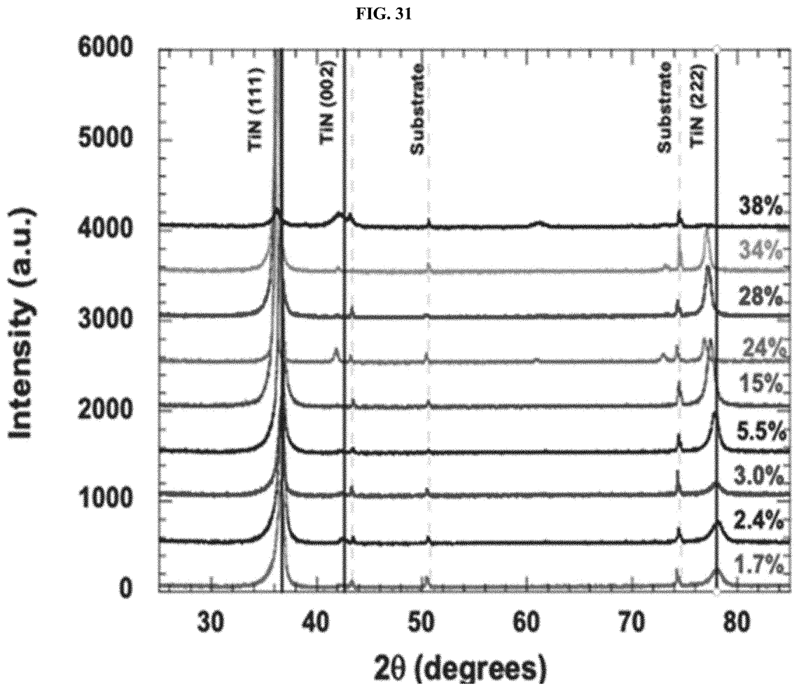

FIG. 30 shows representative images of the microstructures of TiN as a function of zinc (Zn) content. The change of microstructure is obvious from the images. In this study it was found that the transition between the desirable columnar microstructure and the spherical microstructure is between 32 and 34% Zn in comparison to titanium (Ti: 68-66%) with a dependence on deposition pressure.

Measurement of the crystal structure by x-ray diffractometry (XRD) is shown in FIG. 31 for the Zn-containing TiN coatings. Notice that the only significant TiN (002) peak is observed in the sample that contains 38% Zn suggesting that the coatings are highly textured at all other concentrations of Zn. This is consistent with the microstructure shown in FIG. 30 where the surface cube corners are observable in all images (including for concentrations not shown) except for the sample containing 38% Zn. By observing the evolution of the TiN (222) peak, which should also reflect the texturing, it has a maximum intensity at approximately 15% Zn content. Based on this observation, the addition of Zn may improve texturing over a range of compositions before the additions become detrimental to the material microstructure and final performance.

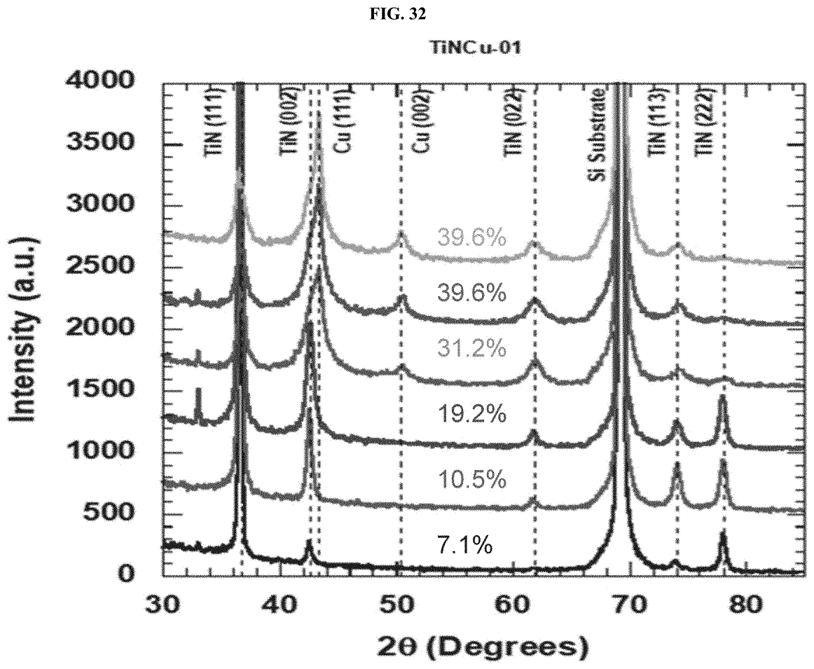

FIG. 32 shows a plot of XRD peaks for TiN containing copper (Cu). At all concentrations shown (7.1% or greater), the TiN (002) peak is observed suggesting the texturing is weaker in the Cu-containing material than it is in the Zn containing coatings. Again, observing the observation of the (222) peak indicates that Cu additions up to approximately 19% may improve the texturing of the coatings.

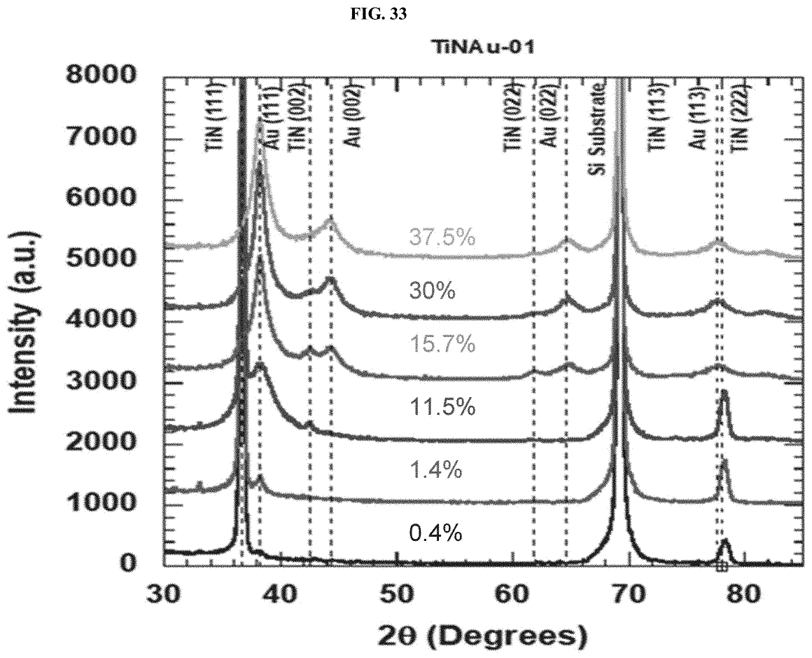

FIG. 33 shows similar data for TiN with gold (Au) additions. Since Au is much heavier than Cu, it is anticipated that it may play a different role in the texturing of the TiN. While the results are qualitatively similar to those observed for the Cu and Zn additions, the Au additions show XRD peaks associated with the Au at fairly low concentrations. In addition, the (222) peak begins to decrease in intensity at Au concentration greater than 11.5%.

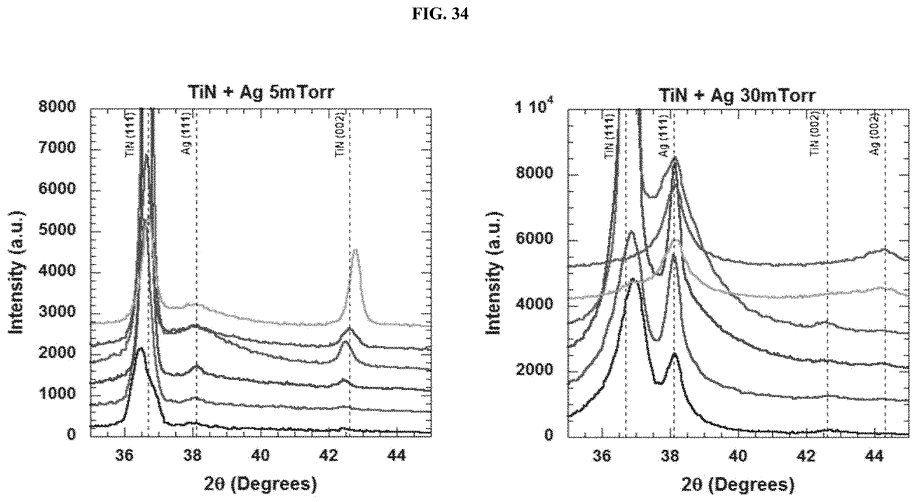

FIG. 34 shows the XRD spectra with silver (Ag) additions for coatings deposited at two different deposition pressures of 5 mTorr and 30 mTorr. Note that the Ag plays a more significant disruptive role in the coatings synthesized at 30 mTorr than it does in those deposited at 5 mTorr. This result indicates that the deposition parameters can be used to optimize coating performance for various concentrations of bactericidal element.

In general, the addition of a bactericidal or impurity element to the TiN eventually impacts the microstructure and the electrochemical performance of the coatings as the concentration increases.

### Example 7: Ion Release

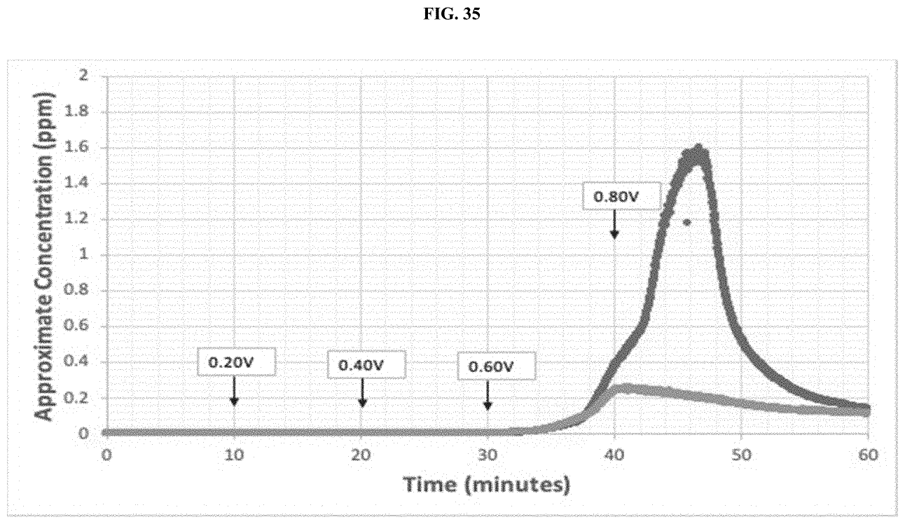

A flow cell apparatus was configured which allowed the application of a potential to the coatings under liquid flow conditions. As the phosphate buffered saline (PBS) solution passed through the cell, when oxidation occurred, ions were released. The output of the cell was passed through a tube into an inductively-coupled plasma mass spectrometer to measure the ion concentration. Since the tube coupling the cell to the spectrometer had a small diameter, there was a delay in the detection of the released ions. To compensate for this effect, the potential was increased in steps of 0.1V per 5-minute time interval. FIG. 35 plots the measured Ag-ion concentration as a function of time. The potentials at various time intervals are placed on FIG. 35 for reference.

This data suggest that release rates can be tailored for the application by adjusting the potential and the initial concentration of the antimicrobial element in the coating. In addition, voltage pulse height and duration will play important roles.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a substrate at least partially coated with a bactericidal coating, the composition comprising:
a substrate having a surface; and
a bactericidal layer comprising a bactericidal metal element and a columnar microstructure, wherein the bactericidal layer comprises at least one of the following:
(a) a multilayer comprising:
a first layer comprising the bactericidal metal element, wherein the first layer is stably adhered to at least one portion of the substrate surface; and
a second layer comprising the columnar microstructure, wherein the second layer is stably adhered to at least one portion of the first layer; or
(b) a composite comprising the bactericidal metal element and the columnar microstructure, wherein the composite is stably adhered to at least one portion of the substrate surface.

2. The composition of claim 1, wherein the bactericidal metal element is at least one selected from the group consisting of Ag, Cu, Zn, Ni, Sn, Au, and Co.

3. The composition of claim 1, wherein the columnar microstructure comprises at least one selected from the group consisting of a metal nitride and a carbon nanotube.

4. The composition of claim 3, wherein the columnar microstructure comprises the metal nitride, and the metal nitride is at least one selected from the group consisting of titanium nitride (TiN), zirconium nitride (ZrN), tantalum nitride (TaN), and vanadium nitride (VN).

5. The composition of claim 1, wherein an amount of the bactericidal metal element ranges from 5% to 18% (mol/mol) based on a total amount of metal elements in the bactericidal layer.

6. The composition of claim 1, wherein the substrate is at least one selected from the group consisting of intramedullary nails, pins, rods, plates, screws, artificial joints, artificial heart components, prosthetic blood vessels, catheters, stents, wound dressings, surgical stitching fibers, and pharmaceutical depots.

7. The composition of claim 1, wherein the bactericidal layer comprises the multilayer, and wherein the multilayer is synthesized by a method comprising DC magnetron sputtering the bactericidal metal element onto at least a portion of the substrate's surface and DC reactive magnetron sputtering the columnar microstructure layer onto a portion of the substrate's surface that is coated by the bactericidal metal element.

8. The composition of claim 1, wherein the bactericidal layer comprises the composite, and wherein the composite bactericidal layer is synthesized by co-deposition of the bactericidal metal element and Ti or Zr onto at least a portion of the substrate's surface with a partial pressure of Na.

9. An electrode article that is implantable in a subject, the article comprising:
an electrode substrate having a surface; and
a bactericidal layer comprising a bactericidal metal element and a columnar microstructure, wherein the bactericidal layer comprises at least one of the following:
(a) a multilayer comprising:
a first layer comprising the bactericidal metal element, wherein the first layer is stably adhered to at least one portion of the substrate surface; and
a second layer comprising the columnar microstructure, wherein the second layer is stably adhered to at least one portion of the first layer; or
(b) a composite comprising the bactericidal metal element and the columnar microstructure, wherein the composite is stably adhered to at least one portion of the substrate surface,
wherein ions of the bactericidal metal element are released by application of an electrical potential to the bactericidal layer.

10. The article of claim 9, wherein the bactericidal metal element is at least one selected from the group consisting of Ag, Cu, Zn, Ni, Sn, Au, and Co.

11. The article of claim 9, wherein the columnar microstructure layer comprises at least one selected from the group consisting of a metal nitride a carbon nanotube.

12. The article of claim 11, wherein the columnar microstructure layer comprises the metal nitride, and wherein the metal nitride is at least one selected from the group consisting of titanium nitride (TiN), zirconium nitride (ZrN), tantalum nitride (TaN), and vanadium nitride (VN).

13. The article of claim 9, wherein an amount of the bactericidal metal element ranges from 5% to 18% (mol/mol) based on a total amount of metal elements in the bactericidal layer.

14. The article of claim 9, wherein the substrate surface comprises titanium or stainless steel.

15. The article of claim 9, wherein the electrode comprises an implantable medical device selected from the group consisting of a pacemaker, cardioverter defibrillator, retinal implant, phrenic nerve stimulator, glucose biosensor, cochlear implant, and an electrical stimulator for pain relief management, Parkinson's disease, and/or epilepsy.

16. The article of claim 9, wherein the bactericidal layer comprises the multilayer, and wherein the multilayer is synthesized by DC magnetron sputtering the bactericidal metal element onto at least a portion of the substrate's surface and DC reactive magnetron sputtering the columnar microstructure layer onto a portion of the substrate's surface that is coated by the bactericidal metal element.

17. The article of claim 9, wherein the bactericidal layer comprises the composite, and wherein the composite is synthesized by co-deposition of the bactericidal metal element and Ti or Zr onto at least a portion of the substrate's surface with a partial pressure of N₂.

18. The article of claim 9, wherein the article further comprises a power source configured to apply the electrical potential to the bactericidal layer.

19. The article of claim 18, wherein the electrical potential applied to the bactericidal layer ranges from about +0.1 V to about +0.8 V.

20. The article of claim 9, wherein the subject is a human.
